(19) United States
(12) Patent Application Publication
(10) Pub. No.: US 2023/0144060 A1
(43) Pub. Date: May 11, 2023

(54) MERS-CoV VACCINE

(71) Applicant: Sumagen Canada Inc., London (CA)

(72) Inventors: Chil-Yong KANG, London (CA); Gyoung Nyoun KIM, London (CA); Kunyu WU, London (CA); Sangkyun LEE, Toronto (CA)

(73) Assignee: Sumagen Canada Inc., London, ON (CA)

(21) Appl. No.: 17/918,244

(22) PCT Filed: Apr. 16, 2021
(86) PCT No.: PCT/CA2021/050514

§ 371 Date: Oct. 11, 2022

Related U.S. Application Data
(60) Provisional application No. 63/011,028, filed on Apr. 16, 2020.

(51) Int. Cl. A61K 39/215; C12N 7/00

(52) U.S. Cl. CPC A61K 39/215; C12N 7/00; C12N 2760/20221; C12N 2770/20034; C12N 2770/20043; C12N 2770/20071; A61K 2039/5256

(57) ABSTRACT

A recombinant vesicular stomatitis vims (rVSV) carrying at least one gene that encodes for a MERS-CoV structural protein or modifications thereof. Vaccines or immunogenic compositions against MERS-CoV, and prime boost immunization platforms a prime boost immunization combination against MERS-CoV including: (a) a prime vaccine or immunogenic composition comprising a rVSV carrying at least one gene that encodes for a MERS-CoV structural protein or modifications thereof, and (b) a boost vaccine or immunogenic composition comprising a rVSV carrying the same at least one gene that encodes for a MERS-CoV structural protein or modifications thereof. The at least one gene can be genetically modified to encode a modified MERS-CoV structural protein that elevates glycoprotein synthesis and trigger efficient humoral immune response.

### REFERENCE TO SEQUENCE LISTING SUBMITTED VIA EFS-WEB

This application includes an electronically submitted sequence listing in .txt format. The .txt file contains a sequence listing entitled “0195924.0007_ST25.txt” created on Apr. 14, 2021 and is 29,211 bytes in size. The sequence listing contained in this .txt file is part of the specification and is hereby incorporated by reference herein in its entirety.

### FIELD OF THE INVENTION

The present invention relates to MERS-CoV, in particular to recombinant vesicular stomatitis viruses containing one or more MERS-CoV structural proteins, vaccines and prime-boost vaccines or immunogenic compositions against MERS-CoV.

### BACKGROUND OF THE INVENTION

Throughout this application, various references are cited in brackets to describe more fully the state of the art to which this invention pertains. The disclosure of these references is hereby incorporated by reference into the present invention.

MERS-CoV is an enveloped, single-stranded, positive-sense RNA virus, which belongs to the β-coronaviruses in the family of Coronaviridae (de Groot et al., 2013). It causes severe acute respiratory disease with symptoms of fever, cough, and shortness of breath in humans, and the fatality reaches as high as 30 to 40% (WHO, 2015). Since the known first cases of the disease in Jordan and Saudi Arabia in 2012, the disease spread to other middle-eastern countries and other parts of the world by travelers. The transmission of the MERS-CoV starts from the dromedary camels to humans, and from human to human transmission occurs through close contacts by the patient to care-takers such as hospital personnel, family members, and other people who are in close contact (Buchholz et al., 2013; Drosten et al., 2013). Considering the high fatality of the disease and the possibility of the epidemics in any parts of the world through human to human contacts, development of an efficient vaccine against MERS-CoV is needed to prevent the onset and the spread of the disease in human. The 3′ one-third of MERS-CoV genome encodes structural proteins such as spike (S) protein, envelope (E) protein, nucleocapsid protein (N), and membrane protein (M) (FIG. 1). All S, E, and M proteins reside on the virus envelope. These three structural proteins form virus-like particles when they are expressed together (Wang et al., 2017). MERS-CoV infects host cells using the spike (S) protein, which is a virus surface glycoprotein forming homotrimers (Kirchdoerfer et al., 2016). S protein is a type I membrane protein, which is cleaved into subunit 1 and subunit 2. Subunit 2 is a transmembrane region and is involved in the fusion activity of S protein to the cellular membrane (Kirchdoerfer et al., 2016; Walls et al., 2016). S protein binds to cellular receptor dipeptidyl peptidase 4 (DPP4) through the receptor binding domain in the Si subunit (Raj et al., 2013). The receptor binding domain (RBD) (FIG. 2) on the spike protein, S, contains a critical neutralizing domain (CND) which generates very effective neutralizing antibodies in vaccinated mice (Lu et al., 2014; Li, 2015; Tai et al., 2017).

An ideal MERS-CoV vaccine should induce completely protective immune responses, must be safe, relatively easy to administrate, and efficient for manufacturing. There is room for an improved MERS-CoV vaccine to meet all the criteria for an ideal MERS-CoV vaccine.

The Applicant has developed a system comprising a combination of vaccines that elicits a response against MERS-CoV.

### SUMMARY OF THE INVENTION

In accordance with the present invention, a recombinant vesicular stomatitis virus (rVSV) carries at least one gene that encodes for a MERS-CoV structural protein or modifications thereof.

In one embodiment of the rVSV of the present invention, the MERS-CoV structural protein or modifications thereof includes one or more of a full-length spike (SF) protein of MERS-CoV, a receptor binding domain (RBD) of the SF protein, an envelope (E) protein of MERS-CoV, or a membrane (M) protein of MERS-CoV, or modifications thereof.

In another embodiment of the rVSV of the present invention, the at least one gene includes a gene that encodes for the RBD having a glycoprotein signal peptide at the NH₂-terminus of the RBD. In one aspect, the glycoprotein signal peptide is a melittin signal peptide (msp).

In another embodiment of the rVSV of the present invention, the at least one gene includes a gene that encodes for the SF protein.

In another embodiment of the rVSV of the present invention, the at least one gene includes a gene that encodes for the E protein.

In another embodiment of the rVSV of the present invention, the at least one gene includes a gene that encodes for the RBD having a glycoprotein signal peptide at the NH₂-terminus of the RBD, a gene that encodes for the E protein and a gene that encodes for the M protein. In one aspect, the glycoprotein signal peptide is a melittin signal peptide (msp).

In another embodiment of the rVSV of the present invention, the at least one gene includes a gene that encodes for the RBD having a glycoprotein signal peptide at the NH₂-terminus of the RBD and a gene that encodes for the E protein. In one aspect the glycoprotein signal peptide is a melittin signal peptide (msp).

In another embodiment of the rVSV of the present invention, the rVSV is a replication competent rVSV of Indiana serotype(rVSV_{Ind}).

In another embodiment of the rVSV of the present invention, the rVSV_{Ind} include a mutant matrix protein gene.

In another embodiment of the rVSV of the present invention, the mutant rVSV_{Ind} matrix protein includes a GML mutation (rVSV_{Ind}-GML).

In another embodiment of the rVSV of the present invention, the rVSV is a replication competent rVSV of New Jersey serotype (rVSV_{NJ}).

In another embodiment of the rVSV of the present invention, the rVSV_{NJ} include a mutant matrix protein gene.

In another embodiment of the rVSV of the present invention, the rVSV_{NJ} M protein includes a

GMM mutation (rVSV_{NJ}-GMM) or a GMML mutation (rVSV_{NJ}-GMML).

In another embodiment, the present invention is a MERS-CoV vaccine or immunogenic composition including a recombinant vesicular stomatitis virus (rVSV) of the present invention.

In one embodiment of the MERS-CoV vaccine or immunogenic composition of the present invention, the MERS-CoV vaccine or immunogenic composition comprises the rVSV carrying the gene that encodes for the full-length spike protein of the MERS-CoV, and wherein the rVSV is rVSV_{Ind}-GML, rVSV_{NJ}-GMM or rVSV_{NJ}-GMML.

In another embodiment of the MERS-CoV vaccine or immunogenic composition of the present invention, the MERS-CoV vaccine or immunogenic composition comprises the rVSV carrying the gene that encodes for the RBD having the glycoprotein signal peptide at the NH₂ terminus of the RBD, and wherein the rVSV is rVSV_{Ind}-GML, rVSV_{NJ}-GMM or rVSV_{NJ}-GMML.

In another embodiment of the MERS-CoV vaccine or immunogenic composition of the present invention, the MERS-CoV vaccine or immunogenic composition comprises the rVSV carrying the gene that encodes for the E protein of the MERS-CoV, and wherein the rVSV is rVSV_{Ind}-GML, rVSV_{NJ}-GMM or rVSV_{NJ}-GMML.

In another embodiment of the MERS-CoV vaccine or immunogenic composition of the present invention, the MERS-CoV vaccine or immunogenic composition comprises the rVSV carrying the gene that encodes for the M protein of the MERS-CoV, and wherein the rVSV is rVSV_{Ind}-GML, rVSV_{NJ}-GMM or rVSV_{NJ}-GMML.

In another embodiment of the MERS-CoV vaccine or immunogenic composition of the present invention, the MERS-CoV vaccine or immunogenic composition comprises the rVSV carrying the gene that encodes for the having the glycoprotein signal peptide at the NH₂-terminus of the RBD and the gene that encodes for the E protein, and wherein the rVSV is rVSV_{Ind}-GML, rVSV_{NJ}-GMM or rVSV_{NJ}-GMML.

In another embodiment of the MERS-CoV vaccine or immunogenic composition of the present invention, the MERS-CoV vaccine or immunogenic composition comprises the rVSV carrying the gene that encodes for the RBD having the glycoprotein signal peptide at the NH₂ terminus of the RBD, the gene that encodes for the E protein of the MERS-CoV and the gene that encodes for the M protein of the MERS-CoV, and wherein the rVSV is rVSV_{Ind}-GML, rVSV_{NJ}-GMM or rVSV_{NJ}-GMML.

In another embodiment of the MERS-CoV vaccine or immunogenic composition of the present invention, the glycoprotein signal peptide is a honeybee melittin signal peptide.

In another embodiment, the present invention is a prime boost immunization combination against MERS-CoV including: (a) a prime vaccine or immunogenic composition comprising a replication competent recombinant vesicular stomatitis virus (rVSV) carrying at least one gene that encodes for a MERS-CoV structural protein or a modification thereof, and (b) a booster vaccine or immunogenic composition comprising a replication competent rVSV carrying the same at least one gene.

In one embodiment of the prime boost immunization combination against MERS-CoV of the present invention, the MERS-CoV structural protein or modification thereof includes one or more of a full-length spike (SF) protein of MERS-CoV, a receptor binding domain (RBD) of the SF protein, an envelope (E) protein of MERS-CoV, or a membrane (M) protein of MERS-CoV, or any modifications thereof.

In another embodiment of the prime boost immunization combination against MERS-CoV of the present invention, the at least one gene includes a gene that encodes for the RBD having a glycoprotein signal peptide at the NH₂-terminus of the RBD.

In another embodiment of the prime boost immunization combination against MERS-CoV of the present invention, the at least one gene includes a gene that encodes for the SF protein.

In another embodiment of the prime boost immunization combination against MERS-CoV of the present invention, the at least one gene includes a gene that encodes for the E protein.

In another embodiment of the prime boost immunization combination against MERS-CoV of the present invention, the at least one gene includes a gene that encodes for the RBD having a glycoprotein signal peptide at the NH₂-terminus of the RBD, the E protein and the M protein.

In another embodiment of the prime boost immunization combination against MERS-CoV of the present invention, the at least one gene includes a gene that encodes for the RBD having a glycoprotein signal peptide at the NH₂-terminus of the RBD and a gene that encodes for the E protein.

In another embodiment of the prime boost immunization combination against MERS-CoV of the present invention, the glycoprotein signal peptide is a honeybee melittin signal peptide.

In another embodiment of the prime boost immunization combination against MERS-CoV of the present invention, the rVSV of the prime vaccine or immunogenic composition and the rVSV of the booster vaccine or immunogenic composition are rVSV of the same serotype.

In another embodiment of the prime boost immunization combination against MERS-CoV of the present invention, the rVSV of the prime vaccine or immunogenic composition and the rVSV of the booster vaccine or immunogenic composition are rVSV of Indiana serotype (rVSV_{Ind}).

In another embodiment of the prime boost immunization combination against MERS-CoV of the present invention, the rVSV of the prime vaccine or immunogenic composition and the rVSV of the booster vaccine or immunogenic composition are rVSV of New Jersey serotype (rVSV_{NJ}).

In another embodiment of the prime boost immunization combination against MERS-CoV of the present invention, the rVSV of the prime vaccine or immunogenic composition is Indiana serotype (VSVInd) and the rVSV of the booster vaccine or immunogenic composition is New Jersey serotype (VSV_{NJ}).

In another embodiment of the prime boost immunization combination against MERS-CoV of the present invention, the rVSV of the prime vaccine or immunogenic composition is New Jersey serotype (rVSV_{NJ}) and the rVSV of the booster vaccine or immunogenic composition is rVSV of Indiana serotype (rVSV_{Ind}).

In another embodiment of the prime boost immunization combination against MERS-CoV of the present invention, the rVSV of the prime vaccine and the rVSV of the booster vaccine include a mutant matrix protein gene of the rVSV.

In another embodiment of the prime boost immunization combination against MERS-CoV of the present invention, when the rVSV is rVSV_{Ind}, the matrix protein of the rVSV_{Ind} includes a GML mutation (rVSV_{Ind}-GML).

In another embodiment of the prime boost immunization combination against MERS-CoV of the present invention, when the rVSV is rVSV_{NJ}, the matrix protein of the rVSV_{NJ} includes a GMM mutation (rVSV_{NJ}-GMM) or a GMML mutation (rVSV_{NJ}-GMML).

In another embodiment of the prime boost immunization combination against MERS-CoV of the present invention, the rVSV of the prime vaccine or immunogenic composition and the rVSV of the booster vaccine or immunogenic composition include are codon optimized for expression in a human cell.

In another embodiment, the present invention is a method for inducing an immune response in a mammal against MERS-CoV, comprising administering to the mammal an effective amount of a vaccine or immunogenic composition of the present invention or administering the mammal a prime boost immunization platform of the present invention.

In one embodiment of the method for inducing an immune response in a mammal against MERS-CoV, the immune response includes a humoral and a cellular immune response.

In another embodiment, the present invention is a use of a MERS-CoV vaccine of the present invention for the prevention or treatment of a MERS-CoV infection.

In another embodiment, the present invention is a use of a combination medicament for the prevention or treatment of a MERS-CoV infection, the combination medicament comprising a prime boost immunization platform of the present invention.

In another embodiment, the present invention is a use of a rVSV of the present invention in the manufacture of a vaccine or immunogenic composition for the prevention or treatment of a MERS-CoV infection.

In accordance to the present invention, a recombinant receptor binding domain (RBD) of a spike protein of MERS-CoV includes or has a honeybee melittin signal peptide (msp) at the NH₂ terminus of the RBD. In one embodiment, said recombinant RBD is encoded by a gene including SEQ ID NO: 20 or consisting essentially of SEQ ID NO: 20 or consisting of SEQ ID NO: 20.

### BRIEF DESCRIPTION OF THE DRAWINGS

The present invention will become more fully understood from the detailed description given herein and from the accompanying drawings, which are given by way of illustration only and do not limit the intended scope of the invention.

FIG. 1. Illustration of a MERS CoV clone.

FIG. 2. (SEQ ID NO: 17) Illustration of MERS CoV spike protein, showing the receptor binding protein.

FIG. 3. Illustration of the generation of an avirulent VSV_{Ind} with mutations in the M gene.

FIG. 4. Illustration of the generation of an avirulent VSV_{NJ} with mutations in the M gene.

FIG. 5. (SEQ ID NO: 17 and 18) Cloning MERS-CoV genes (S, RBD, M, S/E and S/E/M) into rVSV_{Ind}-GML (G21E, M51R, L111A) and rVSV_{NJ}-GMM (G22E, M48R, M51R).

FIG. 6. Illustration of recovery of rVSV by reverse genetics (Buchholz, et al., J. Virol. 73:251, 1999).

FIGS. 7A to 7D. Expression of MERS-CoV proteins (S, RBD, M and E) in three different cell lines (BHK-21 (a), VeroC1008 (b) and Huh-T7 C8 (c)) infected with rVSV_{Ind} series. 7A: rVSV_{Ind}-GML, 2. rVSV_{Ind}-GMLS; 7B: 1. rVSV_{Ind}-GML, 2. rVSV_{Ind}-GML-msp-RBD; 7C: rVSV_{Ind}-GML, 2. rVSV_{Ind}-GML-M; 7D. 1. rVSV_{Ind}-GML, 2. rVSV_{Ind}-GML-E.

FIGS. 8A to 8F. Expression of MERS-CoV proteins (RBD, M and E) in BHK-21infected with rVSV_{NJ}-GMM series carrying the genes that encode these MERS-CoV proteins. Panels 8A, 8B, 8C, 8D, 8E and 8F represent Western blot analyses of all three proteins.

FIGS. 9A to 9C. Detection of MERS-CoV proteins (S, RBD, M and E) in the extracellular culture media of the three different cell lines (BHK-21 (a), VeroC1008 (b) and Huh-T7 C8 (c)) infected with rVSV_{Ind} series. 9A. 1. rVSV_{Ind}-GML, 2. rVSV_{Ind}-GML-S, 3, 4, and 5. rVSV_{Ind}-GML-mspRBD, 9B. 1. rVSV_{Ind}-GML, 2. rVSV_{Ind}-GML-M, 9C. 1. rVSV_{Ind}-GML, 2. rVSV_{Ind}-GLM-E.

FIGS. 10A to 10D. Detection of MERS-CoV proteins (S, RBD, M and E) in pseudotype viral particles from three different cell lines (BHK-21, VeroC1008 and Huh-T7 C8) infected with rVSVNJ-GMM series. 10A. 1. Not infected cells, 2. rVSVInd-GML-S, 10B. 1. Not infected cells, 2. rVSVInd-GML-mspRBD, 10C. 1. Not infected cells, 2. rVSVInd-GML-M, 10D. 1. Not infected cells, 2. rVSVIND-GML-E.

FIGS. 11A to 11E. Electron microphotographs of sedimentable particles in the concentrated culture media from the infected BHK-21 cells. 11A: electron micrograph of control cells; 11B electron micrograph of cells infected with rVSV_{Ind}-GML-S and corresponding Western blot; 11C: electron micrograph of cells infected with rVSV_{Ind}-GML-E and corresponding Western blot; 11D: electron micrograph of cells infected with rVSV_{Ind}-GML-M and corresponding Western blot; 11E: electron micrograph of cells infected with rVSV_{Ind}-GML-EM.

FIG. 12. Illustration of serum antibody titration (MERS groups 1-4 and negative control group).

FIG. 13. Illustration of antibody tier against MERS-CoV S1 RBD in rabbit sera for the different vaccination groups (MERS groups 1-4 and negative control group).

FIGS. 14A to 14B. Plaque reduction in neutralization test. 14A. Neutralizing ability of rabbit sera from groups vaccinated with MERS 1-4 and control (PBS) against MERS-CoV EMC/2012. 14B. Positive neutralizing rabbit monoclonal antibody.

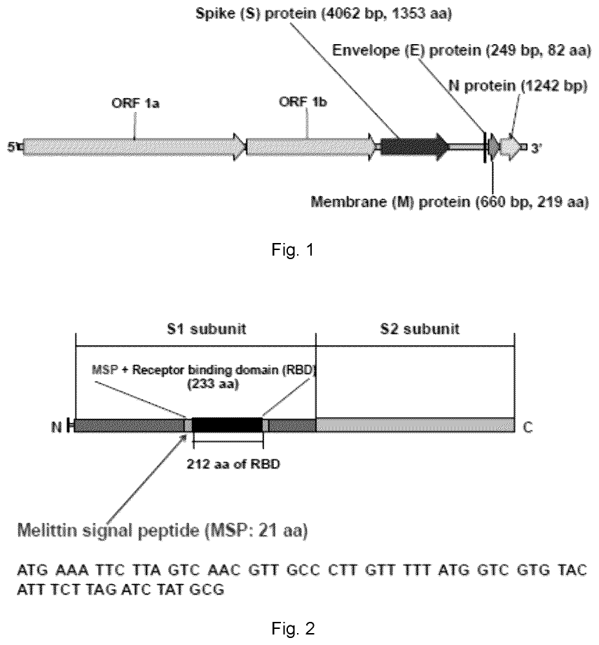

### DETAILED DESCRIPTION OF THE INVENTION

### 1. Definitions

For convenience, the meaning of certain terms and phrases employed in the specification, examples, and appended claims are provided below. Unless defined otherwise, all technical and scientific terms used herein have the same meaning as commonly understood by one of ordinary skill in the art to which this invention belongs.

The articles “a” and “an” are used herein to refer to one or more than one (i.e., to at least one) of the grammatical object of the article.

The terms “animal” and “subject” as used herein includes all members of the animal kingdom including mammals, preferably humans.

The term “effective amount” as used herein means an amount effective and at dosages and for periods of time necessary to achieve the desired result.

“rVSV” is used to refer to a recombinant vesicular stomatitis virus.

The term “Indiana”, and “IND” are used to refer to the VSV serotype Indiana (VSV_{Ind}). The term “New Jersey”, and “NJ” are used to refer to the VSV serotype New Jersey (VSV_{NJ}). In aspects of this invention, the VSV_{NJ} is Hazelhurst strain (VSV_{NJ-H}) or Ogden strain (VSV_{NJ-O}).

“M_{WT}” “M(WT)” are used to refer to VSV expressing a wild type matrix protein.

“G22E” is used to refer to a mutant matrix of VSV_{NJ} having a glycine changed to a glutamic acid at position 22.

“G21E” is used to refer to a mutant matrix protein of VSV_{Ind} having a glycine changed to a glutamic acid at position 21.

“L110A” is used to refer to a mutant matrix protein of VSV_{NJ} having a leucine changed to alanine at position 110.

“L111A” is used to refer to a mutant matrix protein protein of VSV_{Ind} having a leucine changed to alanine at position 111.

“L110F” is used to refer to a mutant matrix protein of VSV_{NJ} having a leucine changed to phenylalanine at position 110.

“L111F” is used to refer to a mutant matrix protein of VSV_{Ind} having a leucine changed to phenylalanine at position 111.

“M51R” is used to refer to mutant matrix protein of the VSV_{Ind} having a methionine changed to an arginine at position 51.

“M48R+M51R” or “M48R/M51R” are used to refer to a mutant matrix protein of VSV_{NJ} having a methionine changed to an arginine at positions 48 and 51 respectively.

“rVSV_{Ind}(GML)” is used to refer to VSV_{Ind} having the combined mutation G21E, M51R and one of L111A or L111F.

“rVSV_{NJ}(GMM)” is used to refer to a VSV_{NJ} having the combined mutation G22E, M48R/M51R.

“rVSV_{NJ}(GMML)” is used to refer to a VSV_{NJ} having the combined mutation G22E, M48R/M51R and one of L110A or L110F.

“S_{F}” is a recombinant full length spike protein of MERS-CoV.

“S protein” is used to refer to the S_{F} or partial length forms of the spike protein of MERS-CoV

“S1” is a recombinant S1 region or subunit of S_{F} of MERS-CoV.

“S2” is a recombinant S2 region or subunit of S_{F} of MERS-CoV.

“RBD” is used to refer to the receptor binding domain of the S_{F}, found in S1 subunit.

“Partial length of the S protein” is used to refer to one or more of S1, S2 and RBD.

The term “protein” as used herein is defined as a chain of amino acid residues, usually having a defined sequence. As used herein the term protein is inclusive of the terms “peptides” and “proteins”. The terms also encompass an amino acid polymer that has been modified.

2. Overview

The present invention features rVSVs, immunization platforms, immunization regimens and medicaments and kits useful for inducing an immune response in a subject and preventing or treating MERS-CoV infection in a subject, wherein said rVSVs, platforms, regimens and medicaments and useful kits comprise a rVSV that carries one or more genes that encode for one or more structural proteins of MERS-CoV, including modifications of said one or more structural proteins to form pseudotype rVSVs that trigger efficient humoral immune responses against MERS-CoV.

The MERS-CoV gene can be genetically modified to encode a modified MERS-CoV structural protein that elevates glycoprotein synthesis and triggers efficient humoral immune response. In one embodiment, the MERS-CoV gene is genetically modified to produce modified structural proteins having a glycoprotein signal peptide at its N-terminus. Any glycoprotein signal peptide that allows the MERS-CoV structural protein to be glycosylated and involved in intracellular trafficking can be used, for example the honeybee melittin signal peptide. For example, a gene is genetically modified to produce RBD proteins having a honeybee melittin signal peptide (msp) at its N-terminus or to produce RBD proteins having the msp at its N-terminus, and the transmembrane domain and cytoplasmic tail of the VSV glycoprotein (Gtc) to form pseudotype VSVs that trigger efficient humoral immune responses against the RBD protein.

In one embodiment, the one or more MERS-CoV structural protein is one or more of a spike (S) protein, a receptor binding domain (RBD) of the S protein, an envelope (E) protein, or a membrane (M) protein of MERS-CoV, modifications of said S, RBD, E and M proteins.

The S protein of MERS-CoV can be a full-length spike (S_{F}) protein or a partial length S protein. The partial length form of the S protein is one or more of S1 peptides of the S_{F} protein, S2 peptides of the S_{F} protein, the receptor binding domain of the S_{F} protein (RBD) or any modifications thereof.

In embodiments, at least one of the S protein (S_{F} or partial length S protein) and the E protein are modified with a glycoprotein signal peptide, such as the honeybee melittin signal peptide (msp), at the NH2-terminus of the at least one of the S protein (S_{F} or partial length S protein) and the E protein, and/or the VSV G protein transmembrane domain and cystoplasmic tail (Gtc) at the COOH-terminus of the at least one of the S protein (S_{F} or partial length S protein) and the E protein.

In one embodiment, the RBD gene is genetically modified to produce an RBD protein having a honeybee melittin signal peptide (msp) at its NH₂-terminus to glycosylate the RBD that trigger efficient humoral immune responses against MERS-CoV.

3. Vaccines or Immunogenic Compositions of the Invention

The present invention further features vaccines or immunogenic compositions.

The present invention describes MERS-CoV vaccines or immunogenic compositions including a recombinant vesicular stomatitis virus (rVSV) that carries one or more genes that encode for at least one MERS-CoV structural protein, including at least one of the S protein (full or partial length forms), the E protein, of MERS-CoV, including modifications of said S, and E proteins. The S protein can be provided as a full-length spike (S_{F}) protein, a S1 subunit of the S_{F} protein, a S2 subunit of the S_{F} protein, and/or a receptor binding domain (RBD) of the S_{F} protein. In embodiments, the at least one of the S (S_{F} or partial length S protein) and E proteins are modified with a glycoprotein signal peptide such as the honeybee melittin signal peptide (msp) at its NH₂-terminus and/or a VSV G protein transmembrane domain and cystoplasmic tail (Gtc) at the COOH-terminus of the S (S_{F} or partial length S protein) and/or E protein. In embodiments, the RBD protein is modified to include a glycoprotein signal peptide, such as the honeybee melittin signal peptide (msp) at its NH₂- terminus to form pseudotype rVSVs that trigger efficient humoral immune responses against MERS-CoV. In embodiments, one or more genes that encode for the S (full or partial length forms), and E proteins and modifications therein are codon-optimized for expression in a human cell. The rVSV may be of Indiana serotype, New Jersey serotype or any other suitable VSV subtype.

The vaccines or immunogenic compositions of this invention may be provided as a prime-boost immunization combination against MERS-CoV. The rVSV of the prime vaccine or immunogenic composition may be of the same or different serotype as the rVSV of the boost vaccine or immunogenic composition. For example, both the prime and boost vaccines or immunogenic compositions are rVSV_{Ind}; or both the prime and boost vaccines or immunogenic compositions are rVSV_{NJ}; or the rVSV of the prime vaccine or immunogenic composition is rVSV_{Ind} and the rVSV of the boost vaccine or immunogenic composition is rVSV_{NJ}; or the rVSV of the prime vaccine or immunogenic composition is rVSV_{NJ} and the rVSV of the boost vaccine or immunogenic composition is rVSV_{Ind}.

The vaccine or immunogenic compositions of the invention are suitable for administration to subjects in a biologically compatible form in vivo. The expression “biologically compatible form suitable for administration in vivo” as used herein means a form of the substance to be administered in which any toxic effects are outweighed by the therapeutic effects. The substances maybe administered to any animal or subject, preferably humans. The vaccines of the present invention may be provided as a lyophilized preparation. The vaccines of the present invention may also be provided as a solution that can be frozen for transportation. Additionally, the vaccines may contain suitable preservatives such as human albumin, bovine albumin, sucrose, glycerol or may be formulated without preservatives. If appropriate (i.e., no damage to the VSV in the vaccine), the vaccines may also contain suitable diluents, adjuvants and/or carriers.

The dose of the vaccine may vary according to factors such as the disease state, age, sex, and weight of the individual, and the ability of antibody to elicit a desired response in the individual. Dosage regime may be adjusted to provide the optimum therapeutic response. The dose of the vaccine may also be varied to provide optimum preventative dose response depending upon the circumstances.

4. Methods of Use

The present invention also features methods of inducing an immune response in a subject against MERS-CoV and/or preventing or treating a MERS-CoV infection in a subject comprising administering to the subject an effective amount of a vaccine or immunogenic composition or a combination of vaccines or immunogenic compositions of the present invention.

As such, in one embodiment, the present invention provides for a method for inducing an immune response in a subject to a MERS-CoV comprising the step (a) of administering to the subject an effective amount of a vaccine or immunogenic composition including a rVSV carrying one or more geneses that encode for one or more structural protein of MERS-CoV. In one embodiment, the method further comprises the step (b) of administering to the subject another vaccine or immunogenic composition comprising a rVSV carrying the same one or more genes that encode the same one or more structural proteins of MERS-CoV.

The rVSV of the vaccine or immunogenic composition of step (a), the priming vaccine or immunogenic composition, may be of the same or different serotype as the rVSV of the vaccine or immunogenic composition (b), the booster vaccine or immunogenic composition. For example, both the prime and boost vaccines or immunogenic compositions are rVSV_{Ind}; or both the prime and boost vaccines or immunogenic compositions are rVSV_{NJ}; or the rVSV of the prime vaccine or immunogenic composition is rVSV_{Ind} and the rVSV of the boost vaccine or immunogenic composition is rVSV_{NJ}; or the rVSV of the prime vaccine or immunogenic composition is rVSV_{NJ} and the rVSV of the boost vaccine or immunogenic composition is rVSV_{Ind}.

In embodiments of the invention the methods for inducing an immune response in a mammal to a MERS-CoV and the methods for preventing or treating an infection caused by MERS-CoV may further comprise the step of (c) administering to the subject an effective amount of the vaccine or immunogenic composition of either step (a) or step (b). Step (c) may be administered to the subject more than one time over the course of inducing an immune response, preventing or treating.

Advantages

Advantages of the recombinant VSV-based platform technology of the present invention are first, a highly efficient prime-boost vaccination can be achieved with two antigenically distinct serotypes of rVSV vectors, because the vector immunity against the priming Indiana serotype (VSV_{Ind}) will not neutralize the boosting New Jersey serotype (VSV_{NJ}) vector. Thus, VSV_{NJ} carrying the same gene of interest as rVSV_{Ind} will provide maximum boost effects. A highly efficient prime-boost vaccination can also be achieved with the same serotype of rVSV vectors (i.e., both the prime and boost are rVSV_{Ind} or both the prime and boost are rVSV_{NJ}), because The pseudotype VSVs carrying both VSV G protein and MERS-CoV spike protein on the surface of the virion can bind to either the low-density lipoprotein receptor (LDL-R) by VSV G protein and/or the human dipeptidyl peptidase 4 (hDPP-4) receptor by the spike protein of MERS-CoV. Thus, the vector immunity against one serotype of VSV may not block the infection of the same pseudotype VSV completely. This may provide boost effects.

Second, the genetically modified VSV_{Ind} M gene mutant (rVSV_{Ind}-GML) and genetically modified VSV_{NJ} M gene mutant (rVSV_{NJ}-GMM) vectors are completely safe, attenuated temperature sensitive mutants [22]. Third, rVSV_{Ind}-GML and rVSV_{NJ}-GMM vectors carrying foreign genes replicate highly efficiently. Therefore, high titer rVSV-based vaccines are relatively easy to prepare. Fourth, both rVSV_{Ind}-GML and rVSV_{NJ}-GMM vectors can accommodate a large-size foreign gene with up to 6,000 nucleotides, without decreasing the virus titer [24], and finally both serotypes of VSV have a very wide host range including humans.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

The examples are described for the purposes of illustration and are not intended to limit the scope of the invention.

We have employed a genetically modified dual serotype of vesicular stomatitis virus (VSV) platform technology [22, 23] to develop vaccines against MERS-CoV.

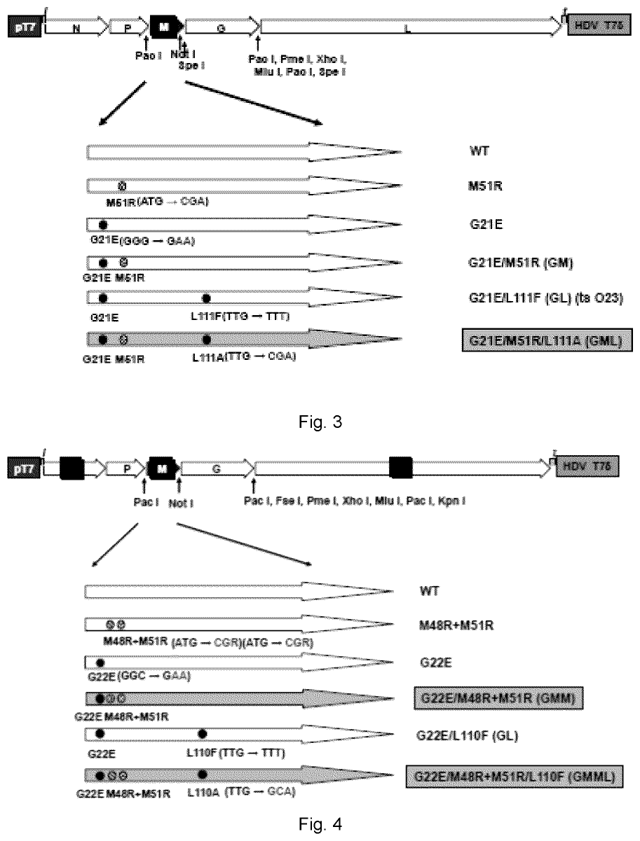

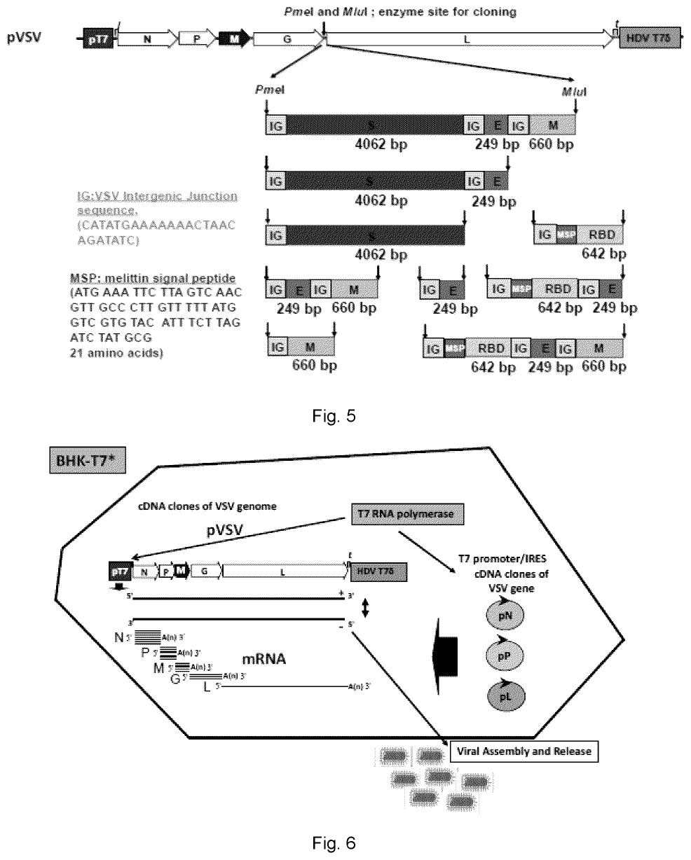

We have developed MERS-CoV vaccines using temperature-sensitive avirulent rVSV_{Ind}-GML and rVSV_{NJ}-GMM as vaccine vectors (FIG. 3, FIG. 4) (Kim et al., 2015). MERS-CoV S, E, and M genes were cloned (FIG. 5), together or separately into the plasmids of rVSV_{Ind}-GML and rVSV_{NJ}-GMM for the generation of the attenuated rVSV vectors expressing MERS-CoV structural proteins. Co-expression of Coronavirus S, E, and M proteins form virus-like particles (VLP) (Boscarino et al., 2008; de Haan et al., 2000; Vennema et al., 1996), which are non-pathogenic and could form a proper conformation of trimeric S proteins on the VLP surface. We also cloned only receptor binding domain (RBD) (FIG. 2, FIG. 5, 212 amino acids) of S protein containing honeybee melittin signal peptide (21 aa, FIG. 5) at the NH2-terminus of RBD (mspRBD) into the VSV vectors. Normally S proteins are secreted as a part of the MERS-CoV particles and stimulate the circulating B lymphocytes to generate neutralizing antibodies against epitopes in the RBD of S protein. However, if RBD is expressed without the signal peptide at the NH2-terminus, the protein would not be secreted outside of the cells resulting in less chance of encountering with B lymphocytes. Naturally, S protein is highly glycosylated in the ER, and lack of signal peptide sequence on the RBD makes the protein non-glycosylated. Signal peptides at the amino-terminal region of the secretory proteins target the protein to the ER and Golgi network for the modification of the protein and to the cytoplasmic membrane for the secretion. Honeybee msp increases the overall expression level, glycosylation, and secretion of the protein through cytoplasmic membrane. Therefore, we added honeybee msp sequences to the NH2-terminus of RBD of S protein to increase the expression of the RBD protein (FIG. 5). We also included E protein and M protein as components of MERS-CoV vaccine together with full length S protein (S or S_{f}) or RBD of S protein and will compare the immunogenicity and efficacy of the vaccine in the presence or absence of E and M proteins.

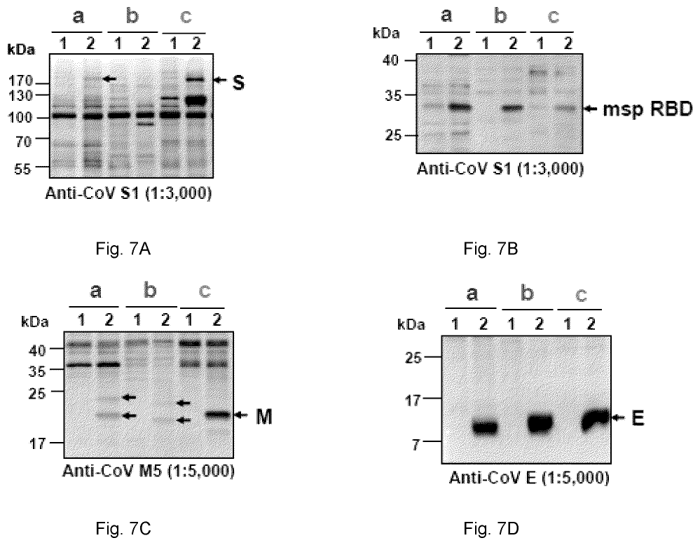

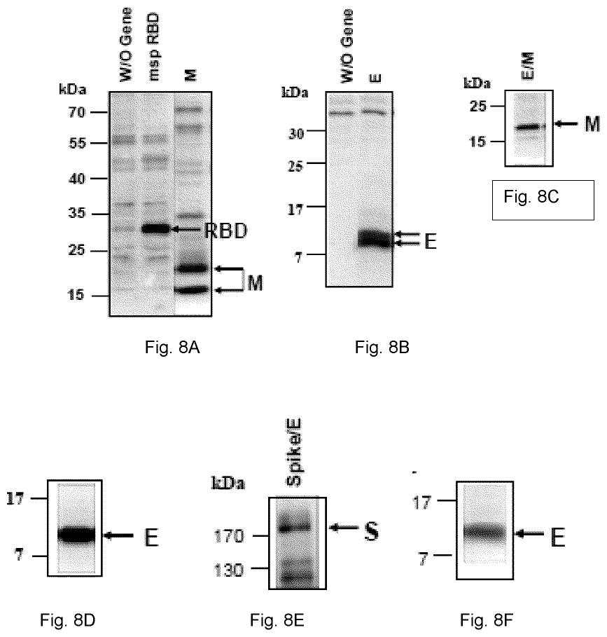

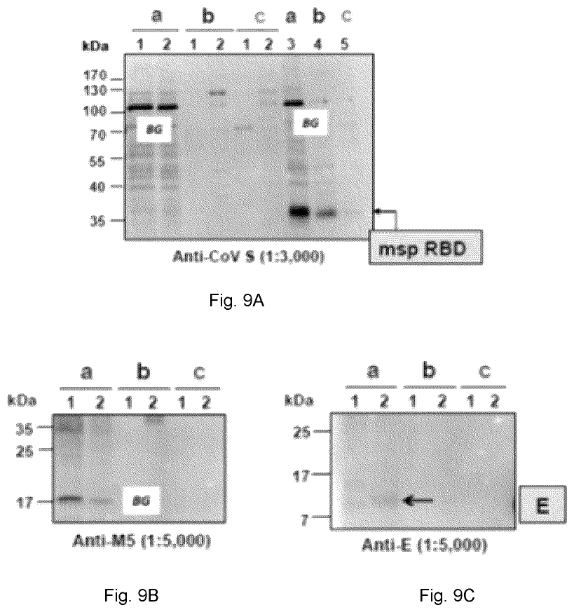

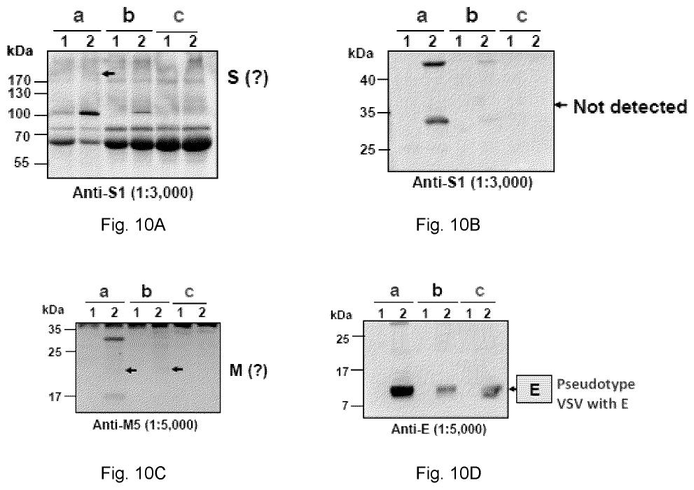

We recovered recombinant VSVs expressing MERS-CoV genes using the VSV reverse genetics system (FIG. 6). The newly recovered viruses are rVSV_{Ind}-GML-S, rVSV_{Ind}-GML-RBD, rVSV_{Ind}-GML-M, rVSV_{Ind}-GML-E, rVSV_{Ind}-GML-E/M, rVSV_{Ind}-GML-S/E, rVSV_{Ind}-GML-mspRBD/E, rVSV_{Ind}-GML-mspRBD/E/M, rVSV_{NJ}-GMM-S, rVSV_{NJ}-GMM-mspRBD, rVSV_{NJ}-GMM-M, rVSV_{NJ}-GMM-E, rVSV_{NJ}-GMM-E/M, rVSV_{NJ}-GMM-S/E, rVSV_{NJ}-GMM-mspRBD/E, and rVSV_{NJ}-GMM-mspRBD/E/M. The recovered viruses were plaque purified three times and amplified in BHK21 cells for virus stock preparation. The intracellular expression of MERS-CoV S, E, and M proteins from the recombinant VSVs were determined by Western blot analysis using rabbit antibodies against S protein (Sino Biological Inc.), rabbit antibodies against E protein (GenScript USA Inc), and rabbit antibodies against M protein (GenScript USA Inc.). Rabbit antibodies against M and E proteins were generated in rabbits using custom-designed linear peptides located at the carboxyl-terminal region of each protein (Table 6, polyclonal antibodies against MERS-CoV Spike protein were purchased from Sino Biological Inc.). BHK2i cells were infected with MOI of 6 of each virus and the cell lysates were prepared at 6 hours post-infection. The 10 μg cell lysates were loaded into the SDS-PAGE gel and MERS-CoV S, RBD, E, and M were detected by Western blot analyses. Proper sizes and good quantities of the MERS-CoV proteins were expressed from the rVSV_{Ind}-GML (FIG. 7) and rVSV_{NJ}-GMM (FIG. 8). About 210 kDa size of S protein was detected from the cells infected with rVSV_{Ind}-GML-S (FIG. 7A), rVSV_{Ind}-GML-S/E (FIG. 7E) and rVSV_{NJ}-GMM-S/E (FIG. 8E). About 30 kDa (212 aa, 23kDa) size of mspRBD was detected from the cells infected with rVSV_{Ind}-GML-mspRBD (FIG. 7A) and rVSV_{NJ}-GMM-mspRBD (FIG. 8A). RBD migrated slightly slower than the actual size RBD probably because of the glycosylation of the protein. E protein was detected as a protein slightly larger than that of 7 kDa protein and showed double bands in a high concentration SDS-PAGE gel (FIG. 7B, 8E, 9B, and 9E), which we are not sure why E protein shows double bands. M protein was detected as about 24 kDa and 22 kDa protein bands when it was expressed in BHK21 cells (FIG. 7A and FIG. 8A). The predicted molecular mass for the M protein is 24 kDa. We are not certain why M protein was detected as 2 separate bands. The differences may come from the differences in the glycosylation or may come from the cleavage of the protein by a cellular protease. We checked the expression of M protein as well as S, RBD, and E proteins in other cell lines such as green monkey kidney cells (Vero) and human liver cells (Huh 7.5) in order to examine whether they are expressed the same as in BHK₂₁ cells. The three different cell lines were infected separately with rVSV_{Ind}-GML-S, rVSV_{Ind}-GML-M, rVSV_{Ind}-GML-mspRBD, rVSV_{Ind}-GML-E (FIG. 9). The infected cells were lysed at 6 hrs post-infection. The expression level and migration pattern in the SDS-PAGE was examined by Western blot analysis (FIG. 9). Full-length S protein was expressed as the same size in all three cell lines, although the expression level was highest in the human liver cell line, Huh7.5 (FIG. 9A line c). M protein was expressed the most in Huh7.5 cells, but the migration pattern was quite different from the M proteins expressed in BHK₂₁ cells and Vero cells (FIG. 9C). M protein expressed in BHK₂₁ cells and Vero cells showed the same migration pattern (FIG. 9C line a and 10C line b). In Huh7.5 cells, M protein migrated as one band, but it migrated faster than the slowly migrating band of the two bands from BHK₂₁ cells and Vero cells (FIG. 9C). It seems that the variability of the M protein expression depends on the origin of the cell lines. We are not certain why M protein shows different expression patterns in different cell lines. The mspRBD and E proteins from three different cell lines showed the same migration pattern (FIG. 9). The expression levels in the three different cell lines were the same for E protein. The mspRBD was expressed the least in Huh7.5 cells (FIG. 9). The expression of VSV proteins in 3 different cell lines did not show much of differences in the level of protein expressions and protein migration patterns in the SDS-PAGE (FIG. 10).

We also checked the secretion of the MERS-CoV proteins from three different cell lines infected with rVSV_{Ind}-GML-S, rVSV_{Ind}-GML-mspRBD, rVSV_{Ind}-GML-M, and rVSV_{Ind}-GML-E (FIG. 10). We wanted to examine whether adding honeybee melittin signal peptide (msp) sequence to the NH2-terminus of the RBD makes it secret or not. The three different cell lines were infected with MOI of 6 and were incubated at 37° C. The culture media from the infected cells were collected at 22 hrs post-infection. The collected culture media was centrifuged at 4,000 rpm for 10 minutes to remove cell debris. The secreted extracellular proteins were concentrated by using ultrafiltration device with 5,000 molecular weight cut-off membrane (Sartorius). The concentrated proteins were detected by Western blot analysis (FIG. 10). MERS-CoV full-length S protein was not detected in the samples from all three cell lines indicating that S does not secret when it is expressed alone without other MERS-CoV proteins (FIG. 10A). On the other hand, mspRBD, which has the msp at the NH2-terminus was secreted from all three different infected cell lines (Fig.10A). The mspRBD was secreted the most from BHK21 cells. Huh7.5 cells secreted the least amount of mspRBD. M protein and E protein was either non-detectable or secreted very small amount in BHK₂₁ cells (FIGS. 10B and 10C) indicating that singly expressed M and E proteins do not secret from the infected cells.

The concentrated culture media contains recombinant VSVs as well as the enveloped structures, which is made of MERS-CoV S, M, or E proteins. Recombinant VSV particles with randomly incorporated MERS-CoV S, E, and M proteins might be present in the concentrated culture media. In order to detect the MERS-CoV S, mspRBD, M, and E proteins, the culture media from the infected cells were collected at 22 hrs post-infection. The collected media was cleared off cell debris and was concentrated by the ultracentrifugation at 36,000 rpm for 2 hrs. MERS-CoV proteins in the pelleted material were detected by Western blot analysis using antibodies against S, M, and E proteins (FIG. 10). Very little amount of S and M protein was detected in samples from BHK₂₁ cells and Vero cells (FIG. 10). There was no detectable amount of S and M proteins in the samples from Huh7.5 cells (FIG. 10). The mspRBD was not present in the pelleted samples from all three different cell lines, indicating that mspRBD alone did not form any sedimentable particles. E protein was detected in all three samples from the different cell lines and BHK21 cells produced the most detectable E protein in the concentrated pellet. The presence of E, M, and S protein in the pelleted samples indicated that there were VSV_{Ind}-GML particles incorporated with these MERS-CoV proteins or sedimented membranous structures with MERS-CoV E, M, and S proteins.

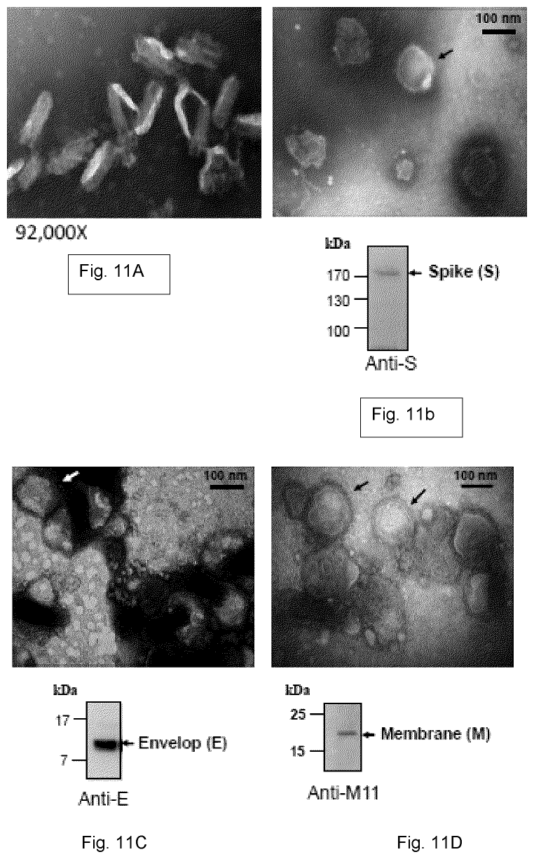

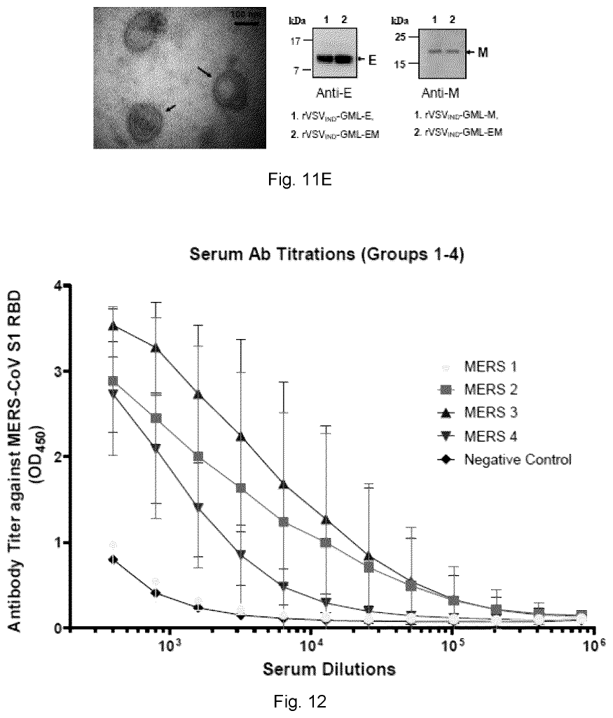

The Western blot analysis using the pelleted culture media indicated that MERS-CoV S, M, and E proteins were part of the sedimentable particles such as virus-like particles (VLP) and/or pseudotyped VSV particles. Therefore, we wanted to examine the presence of such particles which resemble the MERS-CoV by electron microscopy. We infected BHK₂₁ cells separately with MOI of 6 of rVSV_{Ind}-GML-S, rVSV_{Ind}-GML-E, rVSV_{Ind}-GML-M, and rVSV_{Ind}-GML-E/M. The culture media was collected at 22 hrs post-infection. After clearing off cell debris, the culture media was concentrated by ultracentrifugation at 36,000 rpm for 2 hrs. The pelleted material was resuspended in PBS and was fixed in 0.1% glutaraldehyde at room temperature for an hour. The fixed samples were loaded on a formvar/carbon grid and was stained with 1% posphotungstic acid (PTA). All of the samples from BHK₂₁ cells infected with rVSV_{Ind}-GML with S (FIG. 11B), E (FIG. 11C), M (FIG. 11D), or E/M (FIG. 11E) contained typical MERS-CoV structures indicating that as long as one of the structural proteins are expressed, MERS-CoV like membranous structures are generated and are secreted from the infected cells (FIG. 11). The secreted VLPs of MERS-CoV structural proteins will present the antigens to B cells as well as T cells for inducing MERS-CoV protein-specific immune responses. This rVSV-MERS-CoV vaccine has been used for immune response studies.

There is no currently available vaccine against MERS-CoV. Considering the high fatality of the disease, the development of an effective vaccine is required to prevent MERS. Expression of MERS-CoV E, M, and RBD(S) could generate virus-like particles (VLPs) and could induce neutralizing antibodies against MERS-CoV. We generated attenuated rVSVs of both Indiana and New Jersey serotypes expressing MERS-CoV structural proteins, RBD of Spike glycoprotein (S), Envelope protein (E), and Membrane protein (M). The rVSVs of the present invention are noncytolytic and avirulent. We examined the production of neutralization antibodies (nAb) against RBD, a receptor binding domain of Spike glycoprotein. We compared the efficiency of producing nAb by rVSV expressing RBD(S) alone or by rVSV expressing RBD(S), E, and M proteins. We checked the level of nAb against various MERS-CoV isolates. The following animal groups have been vaccinated with rVSV expressing MERS-CoV structural proteins, M, E, and RBD of S proteins. Rabbits were prime-immunized with rVSV_{Ind}-GML expressing MERS-CoV proteins and boos-immunized with rVSV_{NJ}-GMM expressing MERS-CoV proteins (Table 1).

Group 1: As a negative control group, rabbits have been injected with 500 μl of phosphate buffered saline

Group 2: Rabbits have been injected with rVSV without MERS-CoV gene inserts. Each rabbit was prime immunized with 5×10⁸ pfu/500 μl rVSV_{Ind}-GML, 3 weeks after priming, boost immunized with 5×10⁸ pfu/500 μl rVSV_{NJ}-GMM. Two weeks after boost-immunization, rabbits have been euthanized for serum collection

Groups 3: Each rabbit was prime immunized with 5×10⁸ pfu/500 μl rVSV_{Ind}-GML MERS-CoV mspRBD(S), 3 weeks after priming, boost immunized with 5×10⁸ pfu/500 μl rVSV_{NJ}-GMM MERS-CoV mspRBD(S). Two weeks after boost-immunization, rabbits have been euthanized for serum collection.

Groups 4: Each rabbit were prime immunized with 5×10⁸ pfu/500 μl rVSV_{Ind}-GML MERS-CoV mspRBD(S)/E, 3 weeks after priming, boost immunized with 5×10⁸ pfu/500 μl rVSV_{NJ}-GMM MERS-CoV RBD(S)/E. Two weeks after boost-immunization, rabbits have been euthanized for serum collection.

Group 5: Each rabbit was prime immunized with 5×10⁸ pfu/500 μl rVSV_{Ind}-GML MERS-CoV RBD(S)/E/M, 3 weeks after priming, boost immunized with 5×10⁸ pfu/500 μl rVSV_{NJ}-GMM)-N MERS-CoV RBD(S)/E/M. Two weeks after boost-immunization, rabbits have been euthanized for serum collection.

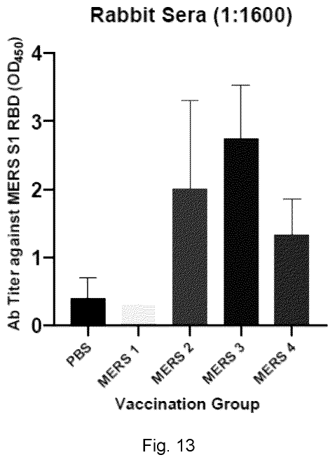

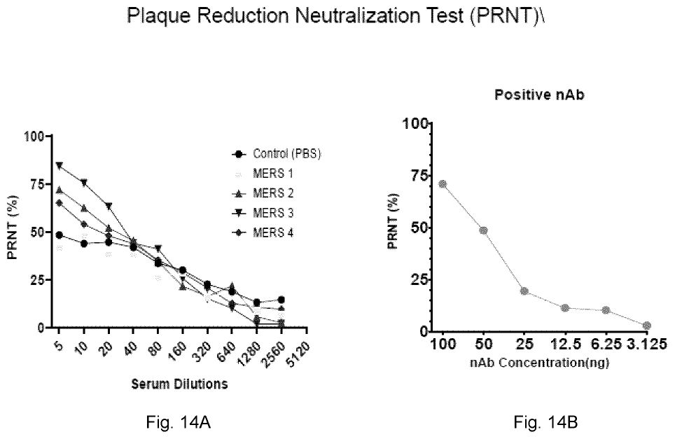

We found rabbits immunized with 5×10⁸ pfu/500 μl of rVSV_{Ind}-GML MERS-CoV mspRBD(S) followed by boost immunization with 5×10⁸ pfu/500 μl rVSV_{NJ}-GMM MERS-CoV mspRBD(S) induced high levels of antibodies (FIG. 12, FIG. 13). Moreover, rabbits immunized with 5×10⁸ pfu/500 μl rVSV_{Ind}-GML MERS-CoV mspRBD(S)/E followed by boost immunization with 5×10⁸ pfu/500 μl rVSV_{NJ}-GMM MERS-CoV RBD(S)/E induced equally high levels of neutralizing antibodies (FIG. 14).

The RBD specific antibodies were generated in rabbits vaccinated with rVSV expressing RBD(S) alone, rVSV expressing RBD(S) and E, and rVSV expressing RBD(S), E, and M (FIG. 12, FIG. 13)). RBD specific antibodies were generated equally well in groups immunized with rVSV expressing RBD(S) alone and rVSV expressing RBD(S) and E, which was shown in the titration curve (FIG. 12, FIG. 13) and in the 1/1600 diluted sera (FIG. 13). We also examined neutralizing ability of the rabbit sera from the vaccinated groups against MERS-CoV EMC/2012 (IVI, Seoul, Korea). The diluted sera were mixed with 200 pfu of MERS-CoV EMC/2012, incubated for 30 min, and the serum-virus mixture was inoculated onto Vero E6 cells. The infected plates were kept in the CO₂ incubator for 3 days until CPE showed 100% in the cells infected with virus only. The results for our rabbit serum samples were compared to a positive neutralizing rabbit monoclonal antibody (Sino Biological, 40069-R723). The rabbit sera from vaccinations with rVSV-MERS-CoV RBD(S) and rVSV-MERS-CoV RBD(S)/E showed 100% to 50% neutralization activity against MERS-CoV EMC/2012 in the dilutions to 1/20 (FIG. 14). Sera from rVSV-MERS-CoV RBD(S)/E vaccination showed better neutralization activity than the sera from rVSV-MERS-CoV RBD(S) (FIG. 14) The results indicated that although the single expression of RBD(S) could induce neutralizing antibodies against MERS-CoV, co-expression of RBD(S) and E protein induce more RBD(S) specific antibodies and neutralize the wild type MERS-CoV EMC/2012 better.

We are now in position to carry out MERS-CoV challenge experiments. There has been advances made in generating small animal models for the MERS-CoV infection. Mice are not susceptible to MERS-CoV infections, however, when human dipeptidyl peptidase 4 (DPP-4), the cell surface receptor for MERS-CoV, were introduced into mouse genome as a transgene, mice became highly susceptible to MERS-CoV infection (Agrawal et al., 2015; Pascal et al., 2015). The infection in the human dipeptidyl peptidase 4 (hDPP-4) transgenic mice, hDPP-4 C57BL/C led to the replication of MERS-CoV in the lung and showed the typical symptoms of MERS-CoV infection, lethargy, rapid and shallow breathing, severe weight loss, and 40-100% mortality. This transgenic mouse is the perfect animal model to test the efficacy of our vaccines against MERS-CoV. We will vaccinate this hDPP-4 transgenic mice with our rVSVs expressing MERS-CoV structural proteins and challenge the vaccinated mice with wild type MERS-CoV and score the clinical signs and mortality of the vaccinated and unvaccinated control mice. We have a new State-Of-The-Art BSL3 Laboratory just opened at Western University where we can carry out the challenge studies using the hDPP-4 C57BL/C transgenic mice. We will use both rVSV_{Ind} and rVSV_{NJ}. In prime-boost vaccines, the priming vaccine or immunogenic composition, are of the same or different serotype as the rVSV of the booster vaccine or immunogenic composition. For example, both the prime and boost vaccines or immunogenic compositions are rVSV_{Ind}; or both the prime and boost vaccines or immunogenic compositions are rVSV_{NJ}; or the rVSV of the prime vaccine or immunogenic composition are rVSV_{Ind} and the rVSV of the boost vaccine or immunogenic composition are rVSV_{NJ}; or the rVSV of the prime vaccine or immunogenic composition are rVSV_{NJ} and the rVSV of the boost vaccine or immunogenic composition will be rVSV_{Ind}.

TABLE 1
Vaccination groups with various vaccines
consisted of MERS-CoV RBS(S), E, and M

| Prime | Boost | | |
|---|---|---|---|
| PBS | PBS | New Zealar [symbol] White | 2.5 kg, 5 |
| rVSV [symbol] (G [symbol] L)-N | rVSV [symbol] (G [symbol] [symbol] )-N | | 2.5 kg, 5 |
| rVSV [symbol] (G [symbol] L)-N-RBD(S) | rVSV [symbol] (G [symbol] [symbol] )-N- | | 2.5 kg, 5 |
| | RBD(S) | | |
| rVSV [symbol] (G [symbol] L)-N- | rVSV [symbol] (G [symbol] [symbol] )-N- | | 2.5 kg, 5 |
| RBD(S) [symbol] E | RBD(S) [symbol] E | | |
| rVSV [symbol] (G [symbol] L)-N- | rVSV [symbol] (G [symbol] [symbol] )-N- | | 2.5 kg, 5 |
| RBD(S) [symbol] E [symbol] [symbol] | RBD(S) [symbol] E [symbol] [symbol] | | |
| | | | 25 |

[symbol]

indicates data missing or illegible when filed

TABLE 2
Neucleotide Sequence Comparison between M Genes of VSV Indiana serotype,
Wild Type (SEQ ID NO: 1) and a Mutant G21E/L111A/M51R (SEQ ID NO: 2)

```
                            1 50
SEQ ID NO: 1: ATGAGTTCCT TAAAGAAGAT TCTCGGTCTG AAGGGGAAAG GTAAGAAATC
SEQ ID NO: 2: ATGAGTTCCT TAAAGAAGAT TCTCGGTCTG AAGGGGAAAG GTAAGAAATC
                            51 100
SEQ ID NO: 1: TAAGAAATTA GGGATCGCAC CACCCCCTTA TGAAGAGGAC ACTAACATGG
SEQ ID NO: 2: TAAGAAATTA GAAATCGCAC CACCCCCTTA TGAAGAGGAC ACTAACATGG
                            101 150
SEQ ID NO: 1: AGTATGCTCC GAGCGCTCCA ATTGACAAAT CCTATTTTGG AGTTGACGAG
SEQ ID NO: 2: AGTATGCTCC GAGCGCTCCA ATTGACAAAT CCTATTTTGG AGTTGACGAG
                            151 200
SEQ ID NO: 1: ATGGACACTC ATGATCCGCA TCAATTAAGA TATGAGAAAT TCTTCTTTAC
SEQ ID NO: 2: CGAGACACTC ATGATCCGCA TCAATTAAGA TATGAGAAAT TCTTCTTTAC
                            201 250
SEQ ID NO: 1: AGTGAAAATG ACGGTTAGAT CTAATCGTCC GTTCAGAACA TACTCAGATG
SEQ ID NO: 2: AGTGAAAA G ACGGTTAGAT CTAATCGTCC GTTCAGAACA TACTCAGATG
                            251 300
SEQ ID NO: 1: TGGCAGCCGC TGTATCCCAT TGGGATCACA TGTACATCGG AATGGCAGGG
SEQ ID NO: 2: TGGCAGCCGC TGTATCCCAT TGGGATCACA TGTACATCGG AATGGCAGGG
                            301 350
SEQ ID NO: 1: AAACGTCCCT TCTACAAGAT CTTGGCTTTT TTGGGTTCTT CTAATCTAAA
SEQ ID NO: 2: AAACGTCCCT TCTACAAGAT CTTGGCTTTT GCAGGTTCTT CTAATCTAAA
                            351 400
SEQ ID NO: 1: GGCCACTCCA GCGGTATTGG CAGATCAAGG TCAACCAGAG TATCACGCTC
SEQ ID NO: 2: GGCCACTCCA GCGGTATTGG CAGATCAAGG TCAACCAGAG TATCACGCTC
                            401 450
SEQ ID NO: 1: ACTGTGAAGG CAGGGCTTAT TTGCCACACA GAATGGGGAA GACCCCTCCC
SEQ ID NO: 2: ACTGTGAAGG CAGGGCTTAT TTGCCACACA GAATGGGGAA GACCCCTCCC
                            451 500
SEQ ID NO: 1: ATGCTCAATG TACCAGAGCA CTTCAGAAGA CCATTCAATA TAGGTCTTTA
SEQ ID NO: 2: ATGCTCAATG TACCAGAGCA CTTCAGAAGA CCATTCAATA TAGGTCTTTA
                            501 550
SEQ ID NO: 1: CAAGGGAACG GTTGAGCTCA CAATGACCAT CTACGATGAT GAGTCACTGG
SEQ ID NO: 2: CAAGGGAACG GTTGAGCTCA CAATGACCAT CTACGATGAT GAGTCACTGG
                            551 600
SEQ ID NO: 1: AAGCAGCTCC TATGATCTGG GATCATTTCA ATTCTTCCAA ATTTTCTGAT
SEQ ID NO: 2: AAGCAGCTCC TATGATCTGG GATCATTTCA ATTCTTCCAA ATTTTCTGAT
                            601 650
SEQ ID NO: 1: TTCAGAGATA AGGCCTTAAT GTTTGGCCTG ATTGTCGAGA AAAAGGCATC
SEQ ID NO: 2: TTCAGAGATA AGGCCTTAAT GTTTGGCCTG ATTGTCGAGA AAAAGGCATC
                            651 700
SEQ ID NO: 1: TGGAGCTTGG GTCCTGGATT CTGTCAGCCA CTTCAAATGA
SEQ ID NO: 2: TGGAGCTTGG GTCCTGGATT CTGTCAGCCA CTTCAAATGA
```

TABLE 3
AmiNO: Acid Sequence Comparison between M Proteins of VSV Indiana
serotype Wild Type (SEQ ID NO: 3) and a Mutant G21E/L111A/M51R
(SEQ ID NO: 4)

```
                            1 21 50
SEQ ID NO: 3: MSSLKKILGL KGKGKKSKKL GIAPPPYEED TNMEYAPSAP IDKSYFGVDE
SEQ ID NO: 4: MSSLKKILGL KGKGKKSKKL EIAPPPYEED TNMEYAPSAP IDKSYFGVDE
                            51 100
SEQ ID NO: 3: MDTHDPHQLR YEKFFFTVKM TVRSNRPFRT YSDVAAAVSH WDHMYIGMAG
SEQ ID NO: 4: RDTHDPHQLR YEKFFFTVKM TVRSNRPFRT YSDVAAAVSH WDHMYIGMAG
                            101 111 150
SEQ ID NO: 3: KRPFYKILAF LGSSNLKATP AVLADQGQPE YHAHCEGRAY LPHRMGKTPP
SEQ ID NO: 4: KRPFYKILAF AGSSNLKATP AVLADQGQPE YHAHCEGRAY LPHRMGKTPP
                            151 200
SEQ ID NO: 3: MLNVPEHFRR PFNIGLYKGT VELTMTIYDD ESLEAAPMIW DHFNSSKFSD
SEQ ID NO: 4: MLNVPEHFRR PFNIGLYKGT VELTMTIYDD ESLEAAPMIW DHFNSSKFSD
                            201 229 250
SEQ ID NO: 3: FRDKALMFGL IVEKKASGAW VLDSVSHFK
SEQ ID NO: 4: FRDKALMFGL IVEKKASGAW VLDSVSHFK
```

TABLE 4
Nucleotide Sequence Comparison between M Genes of VSV New Jersey
serotype Wild Type (SEQ ID NO: 5) and Mutants, G22E/M48R/M51R
(SEQ ID NO: 6) and G22E/L110A/M48R/M51R (SEQ ID NO: 7)

```
                            1 50
SEQ ID NO: 5: ATGAGTTCCT TCAAAAAGAT TCTGGGATTT TCTTCAAAAA GTCACAAGAA
SEQ ID NO: 6: ATGAGTTCCT TCAAAAAGAT TCTGGGATTT TCTTCAAAAA GTCACAAGAA
SEQ ID NO: 7: ATGAGTTCCT TCAAAAAGAT TCTGGGATTT TCTTCAAAAA GTCACAAGAA
                            51 100
SEQ ID NO: 5: ATCAAAGAAA CTAGGCTTGC CACCTCCTTA TGAGGAATCA AGTCCTATGG
SEQ ID NO: 6: ATCAAAGAAA CTAGAATTGC CACCTCCTTA TGAGGAATCA AGTCCTATGG
SEQ ID NO: 7: ATCAAAGAAA CTAGAATTGC CACCTCCTTA TGAGGAATCA AGTCCTATGG
                            101 150
SEQ ID NO: 5: AGATTCAACC ATCTGCCCCA TTATCAAATG ACTTCTTCGG AATGGAGGAT
SEQ ID NO: 6: AGATTCAACC ATCTGCCCCA TTATCAAATG ACTTCTTCGG ACGAGAGGAT
SEQ ID NO: 7: AGATTCAACC ATCTGCCCCA TTATCAAATG ACTTCTTCGG ACGAGAGGAT
                            151 200
SEQ ID NO: 5: ATGGATTTAT ATGATAAGGA CTCCTTGAGA TATGAGAAGT TCCGCTTTAT
SEQ ID NO: 6: CGAGATTTAT ATGATAAGGA CTCCTTGAGA TATGAGAAGT TCCGCTTTAT
SEQ ID NO: 7: CGAGATTTAT ATGATAAGGA CTCCTTGAGA TATGAGAAGT TCCGCTTTAT
                            201 250
SEQ ID NO: 5: GTTGAAGATG ACTGTTAGAG CTAACAAGCC CTTCAGATCG TATGATGATG
SEQ ID NO: 6: GTTGAAGATG ACTGTTAGAG CTAACAAGCC CTTCAGATCG TATGATGATG
SEQ ID NO: 7: GTTGAAGATG ACTGTTAGAG CTAACAAGCC CTTCAGATCG TATGATGATG
                            251 300
SEQ ID NO: 5: TCACCGCAGC GGTATCACAA TGGGATAATT CATACATTGG AATGGTTGGA
SEQ ID NO: 6: TCACCGCAGC GGTATCACAA TGGGATAATT CATACATTGG AATGGTTGGA
SEQ ID NO: 7: TCACCGCAGC GGTATCACAA TGGGATAATT CATACATTGG AATGGTTGGA
                            301 350
SEQ ID NO: 5: AAGCGTCCTT TCTACAAGAT AATTGCTCTG ATTGGCTCCA GTCATCTGCA
SEQ ID NO: 6: AAGCGTCCTT TCTACAAGAT AATTGCTCTG ATTGGCTCCA GTCATCTGCA
SEQ ID NO: 7: AAGCGTCCTT TCTACAAGAT AATTGCTGCA ATTGGCTCCA GTCATCTGCA
                            351 400
SEQ ID NO: 5: AGCAACTCCA GCTGTGTTGG CAGACTTAAA TCAACCAGAG TATTATGCCA
SEQ ID NO: 6: AGCAACTCCA GCTGTGTTGG CAGACTTAAA TCAACCAGAG TATTATGCCA
SEQ ID NO: 7: AGCAACTCCA GCTGTGTTGG CAGACTTAAA TCAACCAGAG TATTATGCCA
                            401 450
SEQ ID NO: 5: CACTAACAGG TCGTTGTTTT CTTCCTCACC GACTCGGATT GATCCCACCG
SEQ ID NO: 6: CACTAACAGG TCGTTGTTTT CTTCCTCACC GACTCGGATT GATCCCACCG
SEQ ID NO: 7: CACTAACAGG TCGTTGTTTT CTTCCTCACC GACTCGGATT GATCCCACCG
                            451 500
SEQ ID NO: 5: ATGTTTAATG TGTCCGAAAC TTTCAGAAAA CCATTCAATA TTGGGATATA
SEQ ID NO: 6: ATGTTTAATG TGTCCGAAAC TTTCAGAAAA CCATTCAATA TTGGGATATA
SEQ ID NO: 7: ATGTTTAATG TGTCCGAAAC TTTCAGAAAA CCATTCAATA TTGGGATATA
                            501 550
SEQ ID NO: 5: CAAAGGGACT CTCGACTTCA CCTTTACAGT TTCAGATGAT GAGTCTAATG
SEQ ID NO: 6: CAAAGGGACT CTCGACTTCA CCTTTACAGT TTCAGATGAT GAGTCTAATG
SEQ ID NO: 7: CAAAGGGACT CTCGACTTCA CCTTTACAGT TTCAGATGAT GAGTCTAATG
                            551 600
SEQ ID NO: 5: AAAAAGTCCC TCATGTTTGG GAATACATGA ACCCAAAATA TCAATCTCAG
SEQ ID NO: 6: AAAAAGTCCC TCATGTTTGG GAATACATGA ACCCAAAATA TCAATCTCAG
SEQ ID NO: 7: AAAAAGTCCC TCATGTTTGG GAATACATGA ACCCAAAATA TCAATCTCAG
                            601 650
SEQ ID NO: 5: ATCCAAAAAG AAGGGCTTAA ATTCGGATTG ATTTTAAGCA AGAAAGCAAC
SEQ ID NO: 6: ATCCAAAAAG AAGGGCTTAA ATTCGGATTG ATTTTAAGCA AGAAAGCAAC
SEQ ID NO: 7: ATCCAAAAAG AAGGGCTTAA ATTCGGATTG ATTTTAAGCA AGAAAGCAAC
                            651 700
SEQ ID NO: 5: GGGAACTTGG GTGTTAGACC AATTGAGTCC GTTTAA
SEQ ID NO: 6: GGGAACTTGG GTGTTAGACC AATTGAGTCC GTTTAA
SEQ ID NO: 7: GGGAACTTGG GTGTTAGACC AATTGAGTCC GTTTAA
```

TABLE 5
AmiNO: Acid Sequence Comparison between M Proteins of VSV New Jersey
serotype Wild Type (SEQ ID NO: 8) and Mutants, G22E/M48R/M51R
(SEQ ID NO: 9) and G22E/L110A/M48R/M51R (SEQ ID NO: 10)

```
                            1 22 50
SEQ ID NO: 8: MSSFKKILGF SSKSHKKSKK LGLPPPYEES SPMEIQPSAP LSNDFFGMED
SEQ ID NO: 9: MSSFKKILGF SSKSHKKSKK LELPPPYEES SPMEIQPSAP LSNDFFGRED
SEQ ID NO: 10:MSSFKKILGF SSKSHKKSKK LELPPPYEES SPMEIQPSAP LSNDFFGRED
                            51 100
SEQ ID NO: 8: MDLYDKDSLR YEKFRFMLKM TVRANKPFRS YDDVTAAVSQ WDNSYIGMVG
SEQ ID NO: 9: RDLYDKDSLR YEKFRFMLKM TVRANKPFRS YDDVTAAVSQ WDNSYIGMVG
SEQ ID NO: 10:RDLYDKDSLR YEKFRFMLKM TVRANKPFRS YDDVTAAVSQ WDNSYIGMVG
                            101 110 150
SEQ ID NO: 8: KRPFYKIIAL IGSSHLQATP AVLADLNQPE YYATLTGRCF LPHRLGLIPP
SEQ ID NO: 9: KRPFYKIIAL IGSSHLQATP AVLADLNQPE YYATLTGRCF LPHRLGLIPP
SEQ ID NO: 10:KRPFYKIIAA IGSSHLQATP AVLADLNQPE YYATLTGRCF LPHRLGLIPP
                            151 200
SEQ ID NO: 8: MFNVSETFRK PFNIGIYKGT LDFTFTVSDD ESNEKVPHVW EYMNPKYQSQ
SEQ ID NO: 9: MFNVSETFRK PFNIGIYKGT LDFTFTVSDD ESNEKVPHVW EYMNPKYQSQ
SEQ ID NO: 10:MFNVSETFRK PFNIGIYKGT LDFTFTVSDD ESNEKVPHVW EYMNPKYQSQ
                            201 250
SEQ ID NO: 8: IQKEGLKFGL ILSKKATGTW VLDQLSPFK
SEQ ID NO: 9: IQKEGLKFGL ILSKKATGTW VLDQLSPFK
SEQ ID NO: 10:IQKEGLKFGL ILSKKATGTW VLDQLSPFK
```

TABLE 6
MERS-CoV E protein (82 aa) (SEQ ID NO: 11)
MLPFVQERIGLFIVNFFIFTVVCAITLLVCMAFLTATRLCV
QCMTGFNTLLVQPALYLYNTGRSVYVKFQDSKPPLPPDEWV
Peptide for antibody (14mer) against MERS-
CoV E protein
SEQ ID NO: 12: FQDSKPPLPPDEWV
MERS-CoV M Protein (219 aa) (SEQ ID NO: 13)
MSNMTQLTEAQIIAIIKDWNFAWSLIFLLITIVLQYGYPSR
SMTVYVFKMFVLWLLWPSSMALSIFSAVYPIDLASQIISGI
VAAVSAMMWISYFVQSIRLFMRTGSWWSFNPETNCLLNVPF
GGTTVVRPLVEDSTSVTAVVTNGHLKMAGMHFGACDYDRLP
NEVTVAKPNVLIALKMVKRQSYGTNSGVAIYHRYKAGNYRS
PPITADIELALLRA
Peptides for antibody against MERS-CoV M
protein (14mer)
SEQ ID NO: 14: TVVRPLVEDSTSVT (M11)
SEQ ID NO: 15: LKMVKRQSYGTNSG (M8)
SEQ ID NO: 16: YKAGNYRSPPITAD (M5)
Melittin Signal Peptide Gene
(SEQ ID NO: 17)
ATG AAA TTC TTA GTC AAC GTT GCC CTT
GTT TTT ATG GTC GTG TAC ATT TCT TAG
ATC TAT GCG
IG:VSV Intergenic Junction sequence
(SEQ ID NO: 18)
CATATGAAAAAAACTAACAGATATC
S Full Length (SF) gene (SEQ ID NO: 19)
GTTTAAACCATATGAAAAAAACTAACAGATATCATGATTCA
CTCTGTGTTCCTGCTGATGTTCCTGCTGACACCAACAGAGT
CCTATGTGGATGTGGGACCTGACTCTGTGAAGTCTGCCTGT
ATTGAGGTGGACATCCAACAGACCTTCTTTGACAAGACCTG
GCCAAGACCAATTGATGTGAGCAAGGCTGATGGCATCATCT
ACCCACAGGGCAGGACCTACAGCAACATCACCATCACCTAC
CAGGGACTGTTTCCATACCAGGGAGATCATGGAGATATGTA
TGTCTACTCTGCTGGTCATGCCACAGGCACCACACCACAGA
AACTGTTTGTGGCTAACTACAGCCAGGATGTGAAGCAGTTT
GCCAATGGCTTTGTGGTGAGGATTGGAGCAGCAGCCAACAG
CACAGGCACAGTGATTATCAGCCCAAGCACCTCTGCCACCA
TCAGGAAGATTTACCCTGCCTTTATGCTGGGCTCCTCTGTG
GGCAACTTCTCTGATGGCAAGATGGGCAGGTTCTTCAACCA
CACCCTGGTGCTGCTGCCTGATGGCTGTGGCACCCTGCTGA
GGGCTTTCTACTGTATCTTGGAACCAAGGTCTGGCAACCAC
TGTCCTGCTGGCAACTCCTACACCTCCTTTGCCACCTACCA
CACACCTGCCACAGACTGTTCTGATGGCAACTACAACAGGA
ATGCCTCCCTGAACTCCTTCAAGGAATACTTCAACCTGAGG
AACTGTACCTTTATGTACACCTACAACATCACAGAGGATGA
GATTTTGGAGTGGTTTGGCATCACCCAGACAGCCCAGGGAG
TGCATCTGTTCTCGAGCAGATATGTGGACCTCTATGGAGGC
AATATGTTCCAGTTTGCCACCCTGCCTGTCTATGACACCAT
CAAATACTACAGCATCATCCCACACAGCATCAGGAGCATCC
AGTCTGACAGGAAGGCTTGGGCTGCCTTCTATGTCTACAAA
CTCCAACCACTGACCTTCCTGCTGGACTTCTCTGTGGATGG
CTACATCAGGAGGGCTATTGACTGTGGCTTCAATGACCTGA
GCCAACTTCACTGTTCCTATGAGTCCTTTGATGTGGAGTCT
GGAGTCTACTCTGTGTCCTCCTTTGAGGCTAAGCCATCTGG
CTCTGTGGTGGAACAGGCTGAGGGAGTGGAGTGTGACTTCA
GCCCACTGCTGTCTGGCACACCTCCACAGGTCTACAACTTC
AAGAGACTGGTGTTCACCAACTGTAACTACAACCTGACCAA
ACTGCTGTCCCTGTTCTCTGTGAATGACTTCACTTGTAGCC
AGATTAGCCCTGCTGCCATTGCCAGCAACTGTTACTCCTCC
CTGATTCTGGACTACTTCTCCTACCCACTGAGTATGAAGTC
TGACCTGTCTGTGTCCTCTGCTGGACCAATCAGCCAGTTCA
ACTACAAGCAGTCCTTCAGCAACCCAACTTGTCTGATTCTG
GCTACAGTGCCACACAACCTGACCACCATCACCAAGCCACT
GAAATACTCCTACATCAACAAGTGTAGCAGACTGCTGTCTG
ATGACAGGACAGAGGTGCCACAACTAGTGAATGCCAACCAA
TACAGCCCATGTGTGAGCATTGTGCCAAGCACAGTGTGGGA
GGATGGAGACTACTACAGGAAGCAACTTAGCCCATTGGAGG
GAGGAGGCTGGCTGGTGGCATCTGGCAGCACAGTGGCTATG
ACAGAACAACTCCAAATGGGCTTTGGCATCACAGTCCAATA
TGGCACAGACACCAACTCTGTGTGTCCAAAATTGGAGTTTG
CCAATGACACCAAGATTGCCAGCCAACTTGGCAACTGTGTG
GAATACTCCCTCTATGGAGTGTCTGGCAGGGGAGTGTTCCA
GAACTGTACTGCTGTGGGAGTGAGACAACAGAGGTTTGTCT
ATGATGCCTACCAGAACCTGGTGGGCTACTACTCTGATGAT
GGCAACTACTACTGTCTGAGGGCTTGTGTGTCTGTGCCTGT
GTCTGTGATTTATGACAAGGAGACCAAGACCCATGCCACCC
TGTTTGGCTCTGTGGCTTGTGAACACATCTCCAGCACAATG
AGTCAATACAGCAGGAGCACCAGGAGTATGCTGAAAAGGAG
GGACAGCACATATGGACCACTCCAAACACCTGTGGGCTGTG
TGCTGGGACTGGTGAACTCCTCCCTGTTTGTGGAGGACTGT
AAACTGCCACTGGGACAATCCCTGTGTGCCCTGCCTGACAC
ACCAAGCACCCTGACACCAAGGTCTGTGAGGTCTGTGCCTG
GAGAGATGAGACTGGCAAGCATTGCCTTCAACCACCCAATC
CAGGTGGACCAACTTAACTCCTCCTACTTCAAACTGAGCAT
CCCAACCAACTTCTCCTTTGGAGTGACCCAGGAATACATCC
AGACCACCATCCAGAAGGTGACAGTGGACTGTAAGCAATAT
GTGTGTAATGGCTTCCAGAAGTGTGAACAACTTCTGAGGGA
ATATGGACAATTCTGTAGCAAGATAAACCAGGCTCTTCATG
GAGCCAACCTGAGACAGGATGACTCTGTGAGGAACCTGTTT
GCCTCTGTGAAGTCCAGCCAGTCCAGCCCAATCATCCCTGG
CTTTGGAGGAGACTTCAACCTGACCCTGTTGGAACCGGTGA
GCATCAGCACAGGCAGCAGGTCTGCCAGGTCTGCCATTGAG
GACCTGCTGTTTGACAAGGTGACCATTGCTGACCCTGGCTA
TATGCAGGGCTATGATGACTGTATGCAACAGGGACCTGCCT
CTGCCAGGGACCTGATTTGTGCCCAATATGTGGCTGGCTAC
AAGGTGCTGCCTCCACTGATGGATGTGAATATGGAGGCTGC
CTACACCTCCTCCCTGCTGGGCAGCATTGCTGGAGTGGGCT
GGACTGCAGGACTGTCCTCCTTTGCTGCCATCCCATTTGCC
CAGAGCATCTTCTACAGACTGAATGGAGTGGGCATCACCCA
ACAGGTGCTGTCTGAGAACCAGAAACTGATTGCCAACAAGT
TCAACCAGGCTCTGGGAGCTATGCAGACAGGCTTCACCACC
ACCAATGAGGCTTTCCAGAAGGTCCAGGATGCTGTGAACAA
CAATGCCCAGGCTCTGAGCAAACTGGCATCTGAACTGAGCA
ACACCTTTGGAGCCATCTCTGCTAGCATTGGAGACATCATC
CAGAGACTGGATGTGTTGGAACAGGATGCCCAGATTGACAG
ACTGATAAATGGCAGACTGACCACCCTGAATGCCTTTGTGG
CTCAACAACTTGTGAGGTCTGAGTCTGCTGCCCTGTCTGCC
CAACTTGCCAAGGACAAGGTGAATGAGTGTGTGAAGGCTCA
AAGCAAGAGGTCTGGCTTCTGTGGACAAGGCACCCACATTG
TGTCCTTTGTGGTGAATGCCCCAAATGGACTCTACTTTATG
CATGTGGGCTACTACCCAAGCAACCACATTGAGGTGGTGTC
TGCCTATGGACTGTGTGATGCTGCCAACCCAACCAACTGTA
TTGCCCCTGTGAATGGCTACTTCATCAAGACCAACAACACC
AGGATTGTGGATGAGTGGTCCTACACAGGCTCCTCCTTCTA
TGCCCCTGAACCAATCACCTCCCTGAACACCAAATATGTGG
CTCCACAGGTGACCTACCAGAACATCAGCACCAACCTGCCT
CCTCCACTGCTGGGCAACAGCACAGGCATTGACTTCCAGGA
TGAACTGGATGAGTTCTTCAAGAATGTGAGCACCAGCATCC
CAAACTTTGGCTCCCTGACCCAGATAAACACCACCCTGCTG
GACCTGACCTATGAGATGCTGTCCCTCCAACAGGTGGTGAA
GGCTCTGAATGAGTCCTACATTGACCTGAAAGAACTGGGCA
ACTACACCTACTACAACAAGTGGCCATGGTACATCTGGCTG
GGCTTCATCGCTGGCCTGGTGGCCCTGGCGCTGTGCGTGTT
CTTCATCCTGTGCTGCACCGGCTGCGGCACCAACTGCATGG
GCAAGCTGAAGTGCAACAGGTGCTGCGACAGGTACGAGGAG
TACGACCTGGAGCCCCACAAGGTGCACGTACATTAAACGCG
T
Msp-RBD (SEQ ID NO: 20)
GTTTAAACCATATGAAAAAAACTAACAGATATCATGAAATT
CTTAGTCAACGTTGCCCTTGTTTTTATGGTCGTGTACATTT
CTTACATCTATGCGCAGGCTGAGGGAGTGGAGTGTGACTTC
AGCCCACTGCTGTCTGGCACACCTCCACAGGTCTACAACTT
CAAGAGACTGGTGTTCACCAACTGTAACTACAACCTGACCA
AACTGCTGTCCCTGTTCTCTGTGAATGACTTCACTTGTAGC
CAGATTAGCCCTGCTGCCATTGCCAGCAACTGTTACTCCTC
CCTGATTCTGGACTACTTCTCCTACCCACTGAGTATGAAGT
CTGACCTGTCTGTGTCCTCTGCTGGACCAATCAGCCAGTTC
AACTACAAGCAGTCCTTCAGCAACCCAACTTGTCTGATTCT
GGCTACAGTGCCACACAACCTGACCACCATCACCAAGCCAC
TGAAATACTCCTACATCAACAAGTGTAGCAGACTGCTGTCT
GATGACAGGACAGAGGTGCCACAACTAGTGAATGCCAACCA
ATACAGCCCATGTGTGAGCATTGTGCCAAGCACAGTGTGGG
AGGATGGAGACTACTACAGGAAGCAACTTAGCCCATTGGAG
GGAGGAGGCTGGCTGGTGGCATCTGGCAGCACAGTGGCTAT
GACAGAACAACTCCAAATGGGCTTTGGCATCACAGTCCAAT
ATGGCACAGACACCAACTCTGTGTGTCCAAAATTGTAA
MERS-CoV E Gene (SEQ ID NO: 21)
GTTTAAACCATATGAAAAAAACTAACAGATATCATGTTACC
CTTTGTCCAAGAACGAATAGGGTTGTTCATAGTAAACTTTT
TCATTTTTACCGTAGTATGTGCTATAACACTCTTGGTGTGT
ATGGCTTTCCTTACGGCTACTAGATTATGTGTGCAATGTAT
GACAGGCTTCAATACCCTGTTAGTTCAGCCCGCATTATACT
TGTATAATACTGGACGTTCAGTCTATGTAAAATTCCAGGAT
AGTAAACCCCCTCTACCACCTGACGAGTGGGTTTAACCCGG
GACGCGT
MERS-CoV M Gene (SEQ ID NO: 22)
GTTTAAACCATATGAAAAAAACTAACAGATATCATGTCTAA
TATGACGCAACTCACTGAGGCGCAGATTATTGCCATTATTA
AAGACTGGAACTTTGCATGGTCCCTGATCTTTCTCTTAATT
ACTATCGTACTACAGTATGGATACCCATCCCGTAGTATGAC
TGTCTATGTCTTTAAAATGTTTGTTTTATGGCTCCTATGGC
CATCTTCCATGGCGCTATCAATATTTAGCGCCGTTTATCCA
ATTGATCTAGCTTCCCAGATAATCTCTGGCATTGTAGCAGC
TGTTTCAGCTATGATGTGGATTTCCTACTTTGTGCAGAGTA
TCCGGCTGTTTATGAGAACTGGATCATGGTGGTCATTCAAT
CCTGAGACTAATTGCCTTTTGAACGTTCCATTTGGTGGTAC
AACTGTCGTACGTCCACTCGTAGAGGACTCTACCAGTGTAA
CTGCTGTTGTAACCAATGGCCACCTCAAAATGGCTGGCATG
CATTTCGGTGCTTGTGACTACGACAGACTTCCTAATGAAGT
CACCGTGGCCAAACCCAATGTGCTGATTGCTTTAAAAATGG
TGAAGCGGCAAAGCTACGGAACTAATTCCGGCGTTGCCATT
TACCATAGATATAAGGCAGGTAATTACAGGAGTCCGCCTAT
TACGGCGGATATTGAACTTGCATTGCTTCGAGCTTAGACGC
GT

### REFERENCES

1. de Groot R J, Baker S C, Baric R S, Brown C S, Drosten C et al. Middle East respiratory syndrome coronavirus (MERS-CoV): announcement of the Coronavirus Study Group. J Virol 2013; 87(14):7790-7792.

2. Buchholz U, Muller M A, Nitsche A, Sanewski A, Wevering N et al. Contact investigation of a case of human novel coronavirus infection treated in a German hospital, October-November 2012. Euro Surveill 2013; 18(8).

3. Drosten C, Seilmaier M, Gorman V M, Hartmann W, Scheible G et al. Clinical features and virological analysis of a case of Middle East respiratory syndrome coronavirus infection. Lancet Infect Dis 2013; 13 (9): 745-751.

4. Wang C, Zheng X, Gai W, Zhao Y, Wang H et al. MERS-CoV virus-like particles produced in insect cells induce specific humoural and cellular imminity in rhesus macaques. Oncotarget 2017; 8(8): 12686-12694.

5. Kirchdoerfer R N, Cottrell C A, Wang N, Pallesen J, Yassine H M et al. Pre-fusion structure of a human coronavirus spike protein. Nature 2016; 531(7592):118-121.

6. Walls A C, Tortorici M A, Bosch B J, Frenz B, Rottier P J M et al. Cryo-electron microscopy structure of a coronavirus spike glycoprotein trimer. Nature 2016; 531(7592):114-117.

7. Raj V S, Mou H, Smits S L, Dekkers D H, Muller M A et al. Dipeptidyl peptidase 4 is a functional receptor for the emerging human coronavirus-EMC. Nature 2013; 495(7440):251-254.

8. Lu L, Liu Q, Zhu Y, Chan K H, Qin L et al. Structure-based discovery of Middle East respiratory syndrome coronavirus fusion inhibitor. Nat Commun 2014; 5:3067.

9. Li F. Receptor recognition mechanisms of coronaviruses: a decade of structural studies. J Virol 2015; 89(4): 1954-1964.

10. Tai W, Wang Y, Fett C A, Zhao G, Li F et al. Recombinant Receptor-Binding Domains of Multiple Middle East Respiratory Syndrome Coronaviruses (MERS-CoVs) Induce Cross-Neutralizing Antibodies against Divergent Human and Camel MERS-CoVs and Antibody Escape Mutants. J Virol 2017; 91(1).

11. Kim, G N, Wu, K, Hong, J P, Awamleh, Z, Kang, C Y. Creation of matrix protein gene variants of two serotypes of vesicular stomatitis virus as prime-boost vaccine vectors. J. Virol 2015; (12) 89:6338-6351.

12. Agrawal A S, Garron T, Tao X, Peng B H, Wakamiya M, Chan T S, et al. Generation of a transgenic mouse model of Middle East respiratory syndrome coronavirus infection and disease. J Virol 2015; 89:3659-70.

13. Pascal K E, Coleman C M, Mujica A O, Kamat V, Badithe A, Fairhurst J, et al. Pre- and postexposure efficacy of fully human antibodies against Spike protein in a novel humanized mouse model of MERS-CoV infection. Proc Natl Acad Sci USA. 2015; 112:8738-43.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this invention.

The invention has been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

1. An avirulent recombinant vesicular stomatitis virus (rVSV) carrying a vesicular stomatitis virus (VSV) G gene and at least one gene that encodes for a MERS-CoV structural protein or modifications thereof.

2. The rVSV of claim 1, wherein the MERS-CoV structural protein or modifications thereof includes one or more of a full-length spike (S_{F}) protein of MERS-CoV, a receptor binding domain (RBD) of the S_{F} protein, an envelope (E) protein of MERS-CoV, or a membrane (M) protein of MERS-CoV, or modifications thereof.

3. The rVSV of claim 1, wherein the at least one gene includes a gene that encodes for a receptor binding domain (RBD) of the spike protein of MERS-CoV (i) having a glycoprotein signal peptide at the NH₂-terminus of the RBD, or (ii) having the glycoprotein signal peptide at the NH₂-terminus of the RBD and a VSV G protein transmembrane domain and cytoplasmic tail (Gtc) at the COOH-terminus of the RBD.

4. The rVSV of claim 2, wherein the at least one gene includes a gene that encodes for the S_{F} protein.

5. The rVSV of claim 2, wherein the at least one gene includes a gene that encodes for the E protein.

6. (canceled)

7. The rVSV of claim 2, wherein the at least one gene includes a gene that encodes for the RBD having a glycoprotein signal peptide at the NH₂-terminus of the RBD and a gene that encodes for the E protein.

8. The rVSV of claim 3, wherein the glycoprotein signal peptide is a melittin signal peptide (msp).

9. The rVSV of claim 1, wherein the rVSV is a replication competent avirulent rVSV of Indiana serotype(rVSV_{Ind}).

10. The rVSV of claim 9, wherein the rVSV_{Ind} includes a mutant matrix protein gene.

11. The rVSV of claim 10, wherein the mutant rVSV_{Ind} matrix protein gene encodes for a matrix protein that includes a GML mutation (rVSV_{Ind}-GML).

12. The rVSV of claim 1, wherein the rVSV is a replication competent avirulent rVSV of New Jersey serotype (rVSV_{NJ}).

13. The rVSV of claim 12, wherein the rVSV_{NJ} includes a mutant matrix protein gene.

14. The rVSV of claim 13, wherein the rVSV_{NJ} M protein gene encodes for a matrix protein that includes a GMM mutation (rVSV_{NJ}-GMM) or a GMML mutation (rVSV_{NJ}-GMML).

15. A MERS-CoV vaccine or immunologenic composition including the avirulent recombinant vesicular stomatitis virus (rVSV) of claim 1.

16. The MERS-CoV vaccine or immunogenic composition of claim 15, wherein the MERS-CoV vaccine or immunogenic composition comprises the avirulent rVSV carrying the gene that encodes for a full-length spike protein of the MERS-CoV, and wherein the avirulent rVSV is rVSV_{Ind}-GML, rVSV_{NJ}-GMM or rVSV_{NJ}-GMML.

17. The MERS-CoV vaccine or immunogenic composition of claim 15, wherein the MERS-CoV vaccine or immunogenic composition comprises the avirulent rVSV carrying the gene that encodes for a RBD having (i) the glycoprotein signal peptide at the NH₂ terminus of the RBD, or (ii) the glycoprotein signal peptide at the NH₂-terminus of the RBD and a VSV G protein transmembrane domain and cytoplasmic tail (Gtc) at the COOH-terminus of the RBD, and wherein the avirulent rVSV is rVSV_{Ind}-GML, rVSV_{NJ}-GMM or rVSV_{NJ}-GMML.

18. (canceled)

19. (canceled)

20. The MERS-CoV vaccine or immunogenic composition of claim 15, wherein the MERS-CoV vaccine or immunogenic composition comprises the avirulent rVSV carrying the gene that encodes for the RBD having (i) the glycoprotein signal peptide at the NH₂-terminus of the RBD or (ii) the glycoprotein signal peptide at the NH₂-terminus of the RBD and a VSV G protein transmembrane domain and cytoplasmic tail (Gtc) at the COOH-terminus of the RBD, and the gene that encodes for the E protein, and wherein the avirulent rVSV is rVSV_{Ind}-GML, rVSV_{NJ}-GMM or rVSV_{NJ}-GMML.

21. (canceled)

22. The MERS-CoV vaccine or immunogenic composition of claim 17, wherein the glycoprotein signal peptide is a honeybee melittin signal peptide.

23. A prime boost immunization combination against MERS-CoV including: (a) a prime vaccine or immunogenic composition comprising a replication competent avirulent recombinant vesicular stomatitis virus (rVSV) carrying a vesicular stomatitis virus (VSV) G gene and at least one gene that encodes for a MERS-CoV structural protein or a modification thereof, and (b) a booster vaccine or immunogenic composition comprising a replication competent avirulent rVSV carrying the VSV G gene and the same at least one gene that encodes for the MERS-CoV structural protein or modification thereof.

24. The prime boost immunization combination against MERS-CoV of claim 23, wherein the MERS-CoV structural protein or modification thereof includes one or more of a full-length spike (S_{F}) protein of MERS-CoV, a receptor binding domain (RBD) of the S_{F} protein, an envelope (E) protein of MERS-CoV, or a membrane (M) protein of MERS-CoV, or any modifications thereof.

25. The prime boost immunization combination against MERS-CoV of claim 23, wherein the at least one gene includes a gene that encodes for a receptor binding domain (RBD) of the spike protein of MERS-CoV (i) having a glycoprotein signal peptide at the NH₂-terminus of the RBD or (ii) having the glycoprotein signal peptide at the NH₂-terminus of the RBD and a VSV G protein transmembrane domain and cytoplasmic tail (Gtc) at the COOH-terminus of the RBD.

26. The prime boost immunization platform of claim 23, wherein the at least one gene includes a gene that encodes for a full-length spike protein of MERS-CoV.

27-30. (canceled)

31. The prime boost immunization platform of claim 23, wherein the rVSV of the prime vaccine or immunogenic composition and the rVSV of the booster vaccine or immunogenic composition are rVSV of the same serotype.

32. The prime boost immunization platform of claim 23, wherein at least one of the rVSV of the prime vaccine or immunogenic composition and the rVSV of the booster vaccine or immunogenic composition is rVSV of Indiana serotype (rVSV_{Ind}), or at least one of the rVSV of the prime vaccine or immunogenic composition and the rVSV of the booster vaccine or immunogenic composition is rVSV of New Jersey serotype (rVSV_{NJ}).

33-35. (canceled)

36. The prime boost immunization platform of claim 32, wherein the rVSV of the prime vaccine and the rVSV of the booster vaccine include a mutant matrix protein gene of the rVSV, wherein when the rVSV is the at least one rVSV_{Ind}, the matrix protein of the rVSV_{Ind} includes a GML mutation (rVSV_{Ind}-GML), and when the rVSV is the at least one rVSV_{NJ}, the matrix protein of the rVSV_{NJ} includes a GMM mutation (rVSV_{NJ}-GMM) or a GMML mutation (rVSV_{NJ}-GMML).

37-39. (canceled)

40. A method for inducing an immune response in a mammal against MERS-CoV, comprising administering to the mammal an effective amount of a vaccine or immunogenic composition of claim 15 or administering to the mammal the prime boost immunization platform of claim 23.

41. The method of claim 40, wherein the immune response includes a humoral and a cellular immune response.

42-45. (canceled)

46. The rVSV of claim 3, wherein said RBD having a glycoprotein signal peptide at the NH2-terminus of the RBD is encoded by a gene including SEQ ID NO: 20.


SEQUENCE LISTING (22 sequences)

SEQ ID NO: 1 - DNA, 690 bp, Vesicular stomatitis virus
  misc_feature (1)..(690): M Gene of VSV Indiana serotype, Wild Type
atgagttccttaaagaagattctcggtctgaaggggaaaggtaagaaatctaagaaattagggatcgcaccacccccttatgaagaggacactaacatggagtatgctccgagcgctccaattgacaaatcctattttggagttgacgagatggacactcatgatccgcatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaagatcttggcttttttgggttcttctaatctaaaggccactccagcggtattggcagatcaaggtcaaccagagtatcacgctcactgtgaaggcagggcttatttgccacacagaatggggaagacccctcccatgctcaatgtaccagagcacttcagaagaccattcaatataggtctttacaagggaacggttgagctcacaatgaccatctacgatgatgagtcactggaagcagctcctatgatctgggatcatttcaattcttccaaattttctgatttcagagataaggccttaatgtttggcctgattgtcgagaaaaaggcatctggagcttgggtcctggattctgtcagccacttcaaatga

SEQ ID NO: 2 - DNA, 690 bp, Artificial Sequence
  Synthetic Mutant G21E/L111A/M51R
atgagttccttaaagaagattctcggtctgaaggggaaaggtaagaaatctaagaaattagaaatcgcaccacccccttatgaagaggacactaacatggagtatgctccgagcgctccaattgacaaatcctattttggagttgacgagcgagacactcatgatccgcatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaagatcttggcttttgcaggttcttctaatctaaaggccactccagcggtattggcagatcaaggtcaaccagagtatcacgctcactgtgaaggcagggcttatttgccacacagaatggggaagacccctcccatgctcaatgtaccagagcacttcagaagaccattcaatataggtctttacaagggaacggttgagctcacaatgaccatctacgatgatgagtcactggaagcagctcctatgatctgggatcatttcaattcttccaaattttctgatttcagagataaggccttaatgtttggcctgattgtcgagaaaaaggcatctggagcttgggtcctggattctgtcagccacttcaaatga

SEQ ID NO: 3 - protein, 229 aa, Vesicular stomatitis virus
  misc_feature (1)..(229): M Protein of VSV Indiana serotype Wild Type
MSSLKKILGLKGKGKKSKKLGIAPPPYEEDTNMEYAPSAPIDKSYFGVDEMDTHDPHQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTVELTMTIYDDESLEAAPMIWDHFNSSKFSDFRDKALMFGLIVEKKASGAWVLDSVSHFK

SEQ ID NO: 4 - protein, 229 aa, Artificial Sequence
  Synthetic Mutant G21E/L111A/M51R
MSSLKKILGLKGKGKKSKKLEIAPPPYEEDTNMEYAPSAPIDKSYFGVDERDTHDPHQLRYEKFFFTVKMTVRSNRPFRTYSDVAAAVSHWDHMYIGMAGKRPFYKILAFAGSSNLKATPAVLADQGQPEYHAHCEGRAYLPHRMGKTPPMLNVPEHFRRPFNIGLYKGTVELTMTIYDDESLEAAPMIWDHFNSSKFSDFRDKALMFGLIVEKKASGAWVLDSVSHFK

SEQ ID NO: 5 - DNA, 686 bp, Vesicular stomatitis virus
  misc_feature (1)..(686): M Genes of VSV New Jersey serotype Wild Type
atgagttccttcaaaaagattctgggattttcttcaaaaagtcacaagaaatcaaagaaactaggcttgccacctccttatgaggaatcaagtcctatggagattcaaccatctgccccattatcaaatgacttcttcggaatggaggatatggatttatatgataaggactccttgagatatgagaagttccgctttatgttgaagatgactgttagagctaacaagcccttcagatcgtatgatgatgtcaccgcagcggtatcacaatgggataattcatacattggaatggttggaaagcgtcctttctacaagataattgctctgattggctccagtcatctgcaagcaactccagctgtgttggcagacttaaatcaaccagagtattatgccacactaacaggtcgttgttttcttcctcaccgactcggattgatcccaccgatgtttaatgtgtccgaaactttcagaaaaccattcaatattgggatatacaaagggactctcgacttcacctttacagtttcagatgatgagtctaatgaaaaagtccctcatgtttgggaatacatgaacccaaaatatcaatctcagatccaaaaagaagggcttaaattcggattgattttaagcaagaaagcaacgggaacttgggtgttagaccaattgagtccgtttaa

SEQ ID NO: 6 - DNA, 686 bp, Artificial Sequence
  Synthetic G22E/M48R/M51R
atgagttccttcaaaaagattctgggattttcttcaaaaagtcacaagaaatcaaagaaactagaattgccacctccttatgaggaatcaagtcctatggagattcaaccatctgccccattatcaaatgacttcttcggacgagaggatcgagatttatatgataaggactccttgagatatgagaagttccgctttatgttgaagatgactgttagagctaacaagcccttcagatcgtatgatgatgtcaccgcagcggtatcacaatgggataattcatacattggaatggttggaaagcgtcctttctacaagataattgctctgattggctccagtcatctgcaagcaactccagctgtgttggcagacttaaatcaaccagagtattatgccacactaacaggtcgttgttttcttcctcaccgactcggattgatcccaccgatgtttaatgtgtccgaaactttcagaaaaccattcaatattgggatatacaaagggactctcgacttcacctttacagtttcagatgatgagtctaatgaaaaagtccctcatgtttgggaatacatgaacccaaaatatcaatctcagatccaaaaagaagggcttaaattcggattgattttaagcaagaaagcaacgggaacttgggtgttagaccaattgagtccgtttaa

SEQ ID NO: 7 - DNA, 686 bp, Artificial Sequence
  Synthetic G22E/L110A/M48R/M51R
atgagttccttcaaaaagattctgggattttcttcaaaaagtcacaagaaatcaaagaaactagaattgccacctccttatgaggaatcaagtcctatggagattcaaccatctgccccattatcaaatgacttcttcggacgagaggatcgagatttatatgataaggactccttgagatatgagaagttccgctttatgttgaagatgactgttagagctaacaagcccttcagatcgtatgatgatgtcaccgcagcggtatcacaatgggataattcatacattggaatggttggaaagcgtcctttctacaagataattgctgcaattggctccagtcatctgcaagcaactccagctgtgttggcagacttaaatcaaccagagtattatgccacactaacaggtcgttgttttcttcctcaccgactcggattgatcccaccgatgtttaatgtgtccgaaactttcagaaaaccattcaatattgggatatacaaagggactctcgacttcacctttacagtttcagatgatgagtctaatgaaaaagtccctcatgtttgggaatacatgaacccaaaatatcaatctcagatccaaaaagaagggcttaaattcggattgattttaagcaagaaagcaacgggaacttgggtgttagaccaattgagtccgtttaa

SEQ ID NO: 8 - protein, 229 aa, Vesicular stomatitis virus
  misc_feature (1)..(229): M Proteins of VSV New Jersey serotype Wild Type
MSSFKKILGFSSKSHKKSKKLGLPPPYEESSPMEIQPSAPLSNDFFGMEDMDLYDKDSLRYEKFRFMLKMTVRANKPFRSYDDVTAAVSQWDNSYIGMVGKRPFYKIIALIGSSHLQATPAVLADLNQPEYYATLTGRCFLPHRLGLIPPMFNVSETFRKPFNIGIYKGTLDFTFTVSDDESNEKVPHVWEYMNPKYQSQIQKEGLKFGLILSKKATGTWVLDQLSPFK

SEQ ID NO: 9 - protein, 229 aa, Artificial Sequence
  Synthetic G22E/M48R/M51R
MSSFKKILGFSSKSHKKSKKLELPPPYEESSPMEIQPSAPLSNDFFGREDRDLYDKDSLRYEKFRFMLKMTVRANKPFRSYDDVTAAVSQWDNSYIGMVGKRPFYKIIALIGSSHLQATPAVLADLNQPEYYATLTGRCFLPHRLGLIPPMFNVSETFRKPFNIGIYKGTLDFTFTVSDDESNEKVPHVWEYMNPKYQSQIQKEGLKFGLILSKKATGTWVLDQLSPFK

SEQ ID NO: 10 - protein, 229 aa, Artificial Sequence
  Synthetic G22E/L110A/M48R/M51R
MSSFKKILGFSSKSHKKSKKLELPPPYEESSPMEIQPSAPLSNDFFGREDRDLYDKDSLRYEKFRFMLKMTVRANKPFRSYDDVTAAVSQWDNSYIGMVGKRPFYKIIAAIGSSHLQATPAVLADLNQPEYYATLTGRCFLPHRLGLIPPMFNVSETFRKPFNIGIYKGTLDFTFTVSDDESNEKVPHVWEYMNPKYQSQIQKEGLKFGLILSKKATGTWVLDQLSPFK

SEQ ID NO: 11 - protein, 82 aa, Middle East respiratory syndrome-related coronavirus
  misc_feature (1)..(82): MERS-CoV E protein (82 aa)
MLPFVQERIGLFIVNFFIFTVVCAITLLVCMAFLTATRLCVQCMTGFNTLLVQPALYLYNTGRSVYVKFQDSKPPLPPDEWV

SEQ ID NO: 12 - protein, 14 aa, Artificial Sequence
  Synthetic Peptide for antibody (14mer) against MERS-CoV E protein
FQDSKPPLPPDEWV

SEQ ID NO: 13 - protein, 219 aa, Middle East respiratory syndrome-related coronavirus
  misc_feature (1)..(219): MERS-CoV M Protein (219 aa)
MSNMTQLTEAQIIAIIKDWNFAWSLIFLLITIVLQYGYPSRSMTVYVFKMFVLWLLWPSSMALSIFSAVYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 14 - protein, 14 aa, Artificial Sequence
  Synthetic Peptides for antibody against MERS-CoV M protein (M11)
TVVRPLVEDSTSVT

SEQ ID NO: 15 - protein, 14 aa, Artificial Sequence
  Synthetic Peptides for antibody against MERS-CoV M protein (M8)
LKMVKRQSYGTNSG

SEQ ID NO: 16 - protein, 14 aa, Artificial Sequence
  Synthetic Peptides for antibody against MERS-CoV M protein  (M5)
YKAGNYRSPPITAD

SEQ ID NO: 17 - DNA, 63 bp, Apis mellifera
  misc_feature (1)..(63): Melittin Signal Peptide Gene
atgaaattcttagtcaacgttgcccttgtttttatggtcgtgtacatttcttagatctatgcg

SEQ ID NO: 18 - DNA, 25 bp, Vesicular stomatitis virus
  misc_feature (1)..(25): IGVSV Intergenic Junction sequence
catatgaaaaaaactaacagatatc

SEQ ID NO: 19 - DNA, 4101 bp, Middle East respiratory syndrome-related coronavirus
  misc_feature (1)..(4101): S Full Length (SF) gene
gtttaaaccatatgaaaaaaactaacagatatcatgattcactctgtgttcctgctgatgttcctgctgacaccaacagagtcctatgtggatgtgggacctgactctgtgaagtctgcctgtattgaggtggacatccaacagaccttctttgacaagacctggccaagaccaattgatgtgagcaaggctgatggcatcatctacccacagggcaggacctacagcaacatcaccatcacctaccagggactgtttccataccagggagatcatggagatatgtatgtctactctgctggtcatgccacaggcaccacaccacagaaactgtttgtggctaactacagccaggatgtgaagcagtttgccaatggctttgtggtgaggattggagcagcagccaacagcacaggcacagtgattatcagcccaagcacctctgccaccatcaggaagatttaccctgcctttatgctgggctcctctgtgggcaacttctctgatggcaagatgggcaggttcttcaaccacaccctggtgctgctgcctgatggctgtggcaccctgctgagggctttctactgtatcttggaaccaaggtctggcaaccactgtcctgctggcaactcctacacctcctttgccacctaccacacacctgccacagactgttctgatggcaactacaacaggaatgcctccctgaactccttcaaggaatacttcaacctgaggaactgtacctttatgtacacctacaacatcacagaggatgagattttggagtggtttggcatcacccagacagcccagggagtgcatctgttctcgagcagatatgtggacctctatggaggcaatatgttccagtttgccaccctgcctgtctatgacaccatcaaatactacagcatcatcccacacagcatcaggagcatccagtctgacaggaaggcttgggctgccttctatgtctacaaactccaaccactgaccttcctgctggacttctctgtggatggctacatcaggagggctattgactgtggcttcaatgacctgagccaacttcactgttcctatgagtcctttgatgtggagtctggagtctactctgtgtcctcctttgaggctaagccatctggctctgtggtggaacaggctgagggagtggagtgtgacttcagcccactgctgtctggcacacctccacaggtctacaacttcaagagactggtgttcaccaactgtaactacaacctgaccaaactgctgtccctgttctctgtgaatgacttcacttgtagccagattagccctgctgccattgccagcaactgttactcctccctgattctggactacttctcctacccactgagtatgaagtctgacctgtctgtgtcctctgctggaccaatcagccagttcaactacaagcagtccttcagcaacccaacttgtctgattctggctacagtgccacacaacctgaccaccatcaccaagccactgaaatactcctacatcaacaagtgtagcagactgctgtctgatgacaggacagaggtgccacaactagtgaatgccaaccaatacagcccatgtgtgagcattgtgccaagcacagtgtgggaggatggagactactacaggaagcaacttagcccattggagggaggaggctggctggtggcatctggcagcacagtggctatgacagaacaactccaaatgggctttggcatcacagtccaatatggcacagacaccaactctgtgtgtccaaaattggagtttgccaatgacaccaagattgccagccaacttggcaactgtgtggaatactccctctatggagtgtctggcaggggagtgttccagaactgtactgctgtgggagtgagacaacagaggtttgtctatgatgcctaccagaacctggtgggctactactctgatgatggcaactactactgtctgagggcttgtgtgtctgtgcctgtgtctgtgatttatgacaaggagaccaagacccatgccaccctgtttggctctgtggcttgtgaacacatctccagcacaatgagtcaatacagcaggagcaccaggagtatgctgaaaaggagggacagcacatatggaccactccaaacacctgtgggctgtgtgctgggactggtgaactcctccctgtttgtggaggactgtaaactgccactgggacaatccctgtgtgccctgcctgacacaccaagcaccctgacaccaaggtctgtgaggtctgtgcctggagagatgagactggcaagcattgccttcaaccacccaatccaggtggaccaacttaactcctcctacttcaaactgagcatcccaaccaacttctcctttggagtgacccaggaatacatccagaccaccatccagaaggtgacagtggactgtaagcaatatgtgtgtaatggcttccagaagtgtgaacaacttctgagggaatatggacaattctgtagcaagataaaccaggctcttcatggagccaacctgagacaggatgactctgtgaggaacctgtttgcctctgtgaagtccagccagtccagcccaatcatccctggctttggaggagacttcaacctgaccctgttggaaccggtgagcatcagcacaggcagcaggtctgccaggtctgccattgaggacctgctgtttgacaaggtgaccattgctgaccctggctatatgcagggctatgatgactgtatgcaacagggacctgcctctgccagggacctgatttgtgcccaatatgtggctggctacaaggtgctgcctccactgatggatgtgaatatggaggctgcctacacctcctccctgctgggcagcattgctggagtgggctggactgcaggactgtcctcctttgctgccatcccatttgcccagagcatcttctacagactgaatggagtgggcatcacccaacaggtgctgtctgagaaccagaaactgattgccaacaagttcaaccaggctctgggagctatgcagacaggcttcaccaccaccaatgaggctttccagaaggtccaggatgctgtgaacaacaatgcccaggctctgagcaaactggcatctgaactgagcaacacctttggagccatctctgctagcattggagacatcatccagagactggatgtgttggaacaggatgcccagattgacagactgataaatggcagactgaccaccctgaatgcctttgtggctcaacaacttgtgaggtctgagtctgctgccctgtctgcccaacttgccaaggacaaggtgaatgagtgtgtgaaggctcaaagcaagaggtctggcttctgtggacaaggcacccacattgtgtcctttgtggtgaatgccccaaatggactctactttatgcatgtgggctactacccaagcaaccacattgaggtggtgtctgcctatggactgtgtgatgctgccaacccaaccaactgtattgcccctgtgaatggctacttcatcaagaccaacaacaccaggattgtggatgagtggtcctacacaggctcctccttctatgcccctgaaccaatcacctccctgaacaccaaatatgtggctccacaggtgacctaccagaacatcagcaccaacctgcctcctccactgctgggcaacagcacaggcattgacttccaggatgaactggatgagttcttcaagaatgtgagcaccagcatcccaaactttggctccctgacccagataaacaccaccctgctggacctgacctatgagatgctgtccctccaacaggtggtgaaggctctgaatgagtcctacattgacctgaaagaactgggcaactacacctactacaacaagtggccatggtacatctggctgggcttcatcgctggcctggtggccctggcgctgtgcgtgttcttcatcctgtgctgcaccggctgcggcaccaactgcatgggcaagctgaagtgcaacaggtgctgcgacaggtacgaggagtacgacctggagccccacaaggtgcacgtacattaaacgcgt

SEQ ID NO: 20 - DNA, 735 bp, Artificial Sequence
  Synthetic Msp-RBD
gtttaaaccatatgaaaaaaactaacagatatcatgaaattcttagtcaacgttgcccttgtttttatggtcgtgtacatttcttacatctatgcgcaggctgagggagtggagtgtgacttcagcccactgctgtctggcacacctccacaggtctacaacttcaagagactggtgttcaccaactgtaactacaacctgaccaaactgctgtccctgttctctgtgaatgacttcacttgtagccagattagccctgctgccattgccagcaactgttactcctccctgattctggactacttctcctacccactgagtatgaagtctgacctgtctgtgtcctctgctggaccaatcagccagttcaactacaagcagtccttcagcaacccaacttgtctgattctggctacagtgccacacaacctgaccaccatcaccaagccactgaaatactcctacatcaacaagtgtagcagactgctgtctgatgacaggacagaggtgccacaactagtgaatgccaaccaatacagcccatgtgtgagcattgtgccaagcacagtgtgggaggatggagactactacaggaagcaacttagcccattggagggaggaggctggctggtggcatctggcagcacagtggctatgacagaacaactccaaatgggctttggcatcacagtccaatatggcacagacaccaactctgtgtgtccaaaattgtaa

SEQ ID NO: 21 - DNA, 294 bp, Middle East respiratory syndrome-related coronavirus
  misc_feature (1)..(294): MERS-CoV E Gene
gtttaaaccatatgaaaaaaactaacagatatcatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacccgggacgcgt

SEQ ID NO: 22 - DNA, 699 bp, Middle East respiratory syndrome-related coronavirus
  misc_feature (1)..(699): MERS-CoV M Gene
gtttaaaccatatgaaaaaaactaacagatatcatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttagacgcgt